SEQUENCE LISTING (12 sequences)

SEQ ID NO: 1 - DNA, 25 bp, Artificial Sequence
  actin forward PCR primer
tcctagcaccatgaagatcaagatc

SEQ ID NO: 2 - DNA, 21 bp, Artificial Sequence
  actin reverse PCR primer
ctgcttgctgatccacatctg

SEQ ID NO: 3 - DNA, 20 bp, Artificial Sequence
  S100a9 forward PCR primer
ggaaggaaggacaccctgac

SEQ ID NO: 4 - DNA, 19 bp, Artificial Sequence
  S100a9 reverse PCR primer
ccaggtcctccatgatgtc

SEQ ID NO: 5 - DNA, 20 bp, Artificial Sequence
  Il1f6 forward PCR primer
ctgttctgcacaaaggatgg

SEQ ID NO: 6 - DNA, 21 bp, Artificial Sequence
  Il1f6 reverse PCR primer
gctgcagactcaaatgtagag

SEQ ID NO: 7 - DNA, 18 bp, Artificial Sequence
  Il22ra forward PCR primer
ggacaccccgcttcactc

SEQ ID NO: 8 - DNA, 20 bp, Artificial Sequence
  Il22ra reverse PCR primer
atttggcaactctggaggac

SEQ ID NO: 9 - DNA, 22 bp, Artificial Sequence
  Chi3l1 forward PCR primer
agcagtatttctccaccctgat

SEQ ID NO: 10 - DNA, 20 bp, Artificial Sequence
  Chi3l1 reverse PCR primer
cgctgagcaggagtttctct

SEQ ID NO: 11 - DNA, 20 bp, Artificial Sequence
  Ccl20 forward PCR primer
aactgggtgaaaagggctgt

SEQ ID NO: 12 - DNA, 20 bp, Artificial Sequence
  Ccl20 reverse PCR primer
gtccaattccatcccaaaaa

(19) United States
(12) Patent Application Publication
(10) Pub. No.: US 2013/0102486 A1
(43) Pub. Date: Apr. 25, 2013

(54) PERP AS A PROGNOSTIC AND DIAGNOSTIC MARKER FOR DYSPLASIA AND CANCER

(71) Applicant: The Board of Trustees of the Leland Stanford, Palo Alto, CA (US)

(72) Inventors: Laura D. Attardi, Stanford, CA (US); Quynh-Thu Le, San Carlos, CA (US); Veronica G. Beaudry, Menlo Park, CA (US)

(73) Assignee: The Board of Trustees of the Leland Stanford Junior University, Palo Alto, CA (US)

(21) Appl. No.: 13/656,653

(22) Filed: Oct. 19, 2012

Related U.S. Application Data
(60) Provisional application No. 61/549,727, filed on Oct. 20, 2011.

(51) Int. Cl. G01N 33/574; G01N 33/53; C40B 30/04; C12Q 1/68; G01N 21/76; C40B 40/08

(52) U.S. Cl. 506/9; 435/7.1; 506/17; 435/6.12

(57) ABSTRACT

Methods for prognosis and diagnosis of dysplasia and cancer are disclosed. In particular, the invention relates to the use of the biomarker PERP, a desmosome protein involved in cell adhesion and apoptosis, for aiding diagnosis, prognosis, and treatment of dysplasia and cancer.

### CROSS-REFERENCE TO RELATED APPLICATION

This application claims benefit under 35 U.S.C. §119(e) of provisional application 61/549,727, filed Oct. 20, 2011, which application is hereby incorporated by reference in its entirety.

### STATEMENT REGARDING FEDERALLY SPONSORED RESEARCH OR DEVELOPMENT

This invention was made with Government support under contracts CA119944, CA093665 and CA067166 awarded by the National Institutes of Health. The Government has certain rights in this invention.

### TECHNICAL FIELD

The present invention pertains generally to methods of using biomarkers for diagnosis of cancer. In particular, the invention relates to the use of the biomarker PERP, a desmosome protein involved in cell adhesion and apoptosis, for aiding diagnosis, prognosis, and treatment of cancer and dysplasia.

### BACKGROUND

Normal growth and maintenance of stratified epithelia, such as the epidermis, require the coordinated regulation of cell proliferation, adhesion, migration, and differentiation (Fuchs et al. (2002) Nat. Rev. Genet. 3:199-209). Progenitor cells of the basal, inner layer of the epidermis renew the epidermis by initially proliferating, then exiting the cell cycle, detaching from the basement membrane, migrating, and differentiating to form the upper layers of the skin. The cells of stratified epithelia require a great deal of plasticity to permit division and migration of cells during the differentiation process while still providing a protective barrier to prevent dehydration and infection. This plasticity relies on the modulation of various intercellular adhesion junctions, including tight junctions, adherens junctions and desmosomes (Green et al. (2000) Nat. Rev. Mol. Cell. Biol. 1: 208-216). Loss of adhesion between cells is associated with cancer development and progression, in particular with metastasis involving cell detachment and migration.

Desmosomes help provide epithelia with the strength needed to withstand mechanical stress via anchorage to the intermediate filament network (Green et al., supra; Yin et al. (2004) Semin. Cell. Dev. Biol. 15: 665-677). The crucial nature of desmosomes in maintaining the integrity of stratified epithelia is underscored by the observation that mutation or inactivation of several desmosomal proteins is linked to human epithelial diseases (Chidgey (2002) Histol. Histopathol. 17:1179-1192). PERP (p53 apoptosis effector related to PMP-22) is a desmosome protein involved in the p53 apoptotic pathway and p63 mediated cell-cell adhesion (Fuchs et al., supra; Green et al., supra). Loss of PERP leads to dysregulated cell-cell adhesion by causing decreased numbers of desmosomes and destabilization of desmosome complexes.

Carcinomas, or cancers of epithelia, comprise approximately 90% of all human cancers (Cooper (1995) Oncogenes. Boston: Jones and Bartlett Publishers. XV, p. 384). Cancers of the stratified epithelia, such as the skin and the tissues of the head and neck, are common and often fatal (Samarasinghe et al. (2011) Expert Rev. Anticancer Ther. 11:763-769; Alam et al. (2001) New Engl. J. Med. 344: 975-983; Hunter et al. (2005) Nat. Rev. Cancer 5: 127-135). Early detection coupled with effective therapeutic intervention is crucial for improving chances of survival and extending the lifespan of patients with cancer.

There remains a need in the art for improved methods for diagnosing cancer. The identification of new biomarkers that can detect cancer at an early stage would enable earlier treatment and enhance patient survival.

### SUMMARY

The present invention relates to the use of PERP, a desmosome protein involved in cell adhesion and apoptosis, as a biomarker for aiding diagnosis, prognosis, and treatment of dysplasia and cancer. PERP deficiency is associated with the progression of low grade dysplasia to higher grades of dysplasia and to cancer. The inventors have shown that monitoring PERP levels is useful for identifying patients with increased risk of progression to higher grade disease. The inventors have further shown that PERP loss is linked to tumor aggressiveness and high risk of local relapse (see Examples 1 and 2).

In one aspect, the invention includes a method for diagnosing cancer or dysplasia in a subject. The method comprises (i) measuring the amount of PERP in a biological sample derived from a subject; and (ii) analyzing the amount of PERP and comparing with reference value ranges for PERP, wherein decreased expression of PERP in the biological sample compared to the amount of PERP in a control sample obtained from a healthy individual, who does not have cancer or dysplasia, indicates that the subject has cancer or dysplasia.

PERP can be used as a prognostic or diagnostic biomarker for carcinomas, such as squamous cell carcinoma, adenocarcinoma, adenosquamous carcinoma, anaplastic carcinoma, large cell carcinoma, and small cell carcinoma. In certain embodiments, PERP is used as a prognostic or diagnostic biomarker for skin squamous cell carcinoma, oral cavity squamous cell carcinoma, head squamous cell carcinoma, neck squamous cell carcinoma, and esophageal squamous cell carcinoma.

The biological sample collected from a subject may be a sample of tissue or cells, including but not limited to, samples of skin, organs, or biopsies. For example, the biological sample may include abnormal tissue suspected of containing cancerous or dysplastic cells. The biological sample may also include samples from in vitro cell culture resulting from the growth of cells or tissues from the subject in culture.

In certain embodiments, the amount of PERP is compared with reference value ranges for PERP. The reference value ranges can represent the amount of PERP found in one or more samples of one or more subjects without cancer or dysplasia (i.e., normal samples). Alternatively, the reference value ranges can represent the amount of PERP found in one or more samples of one or more subjects with cancer or dysplasia. More specifically, the reference value ranges can represent the amount of PERP at particular stages of disease (e.g., mild, moderate, or severe dysplasia, cancer in situ, or invasive cancer) to facilitate a determination of the stage of disease progression in an individual.

PERP can be used in combination with other biomarkers including, but not limited to, plakoglobin, desmoglein 1/3, E-cadherin, interleukin 1 family member 6 (IL1f6), S100a9, chitinase 3-like 1 (Chi311), chemokine ligand 20 (Ccl20), and interleukin-22 receptor (IL22ra). In one embodiment, a panel of biomarkers comprising PERP and further comprising one or more polypeptides selected from the group consisting of plakoglobin, desmoglein 1/3, E-cadherin, interleukin 1 family member 6 (IL1f6), S100a9, chitinase 3-like 1 (Chi311), chemokine ligand 20 (Ccl20), and interleukin-22 receptor (IL22ra) is used for diagnosis of cancer or dysplasia.

PERP and other biomarkers can be measured by any suitable method including, but not limited to, immunohistochemistry, enzyme-linked immunosorbent assay (ELISA), radioimmunoassay (RIA), immunofluorescent assay (IFA), sandwich assay, magnetic capture, microsphere capture, Western Blot, flow cytometry, or mass spectrometry. In certain embodiments, the level of a biomarker is measured by contacting an antibody with the biomarker, wherein the antibody specifically binds to the biomarker, or a fragment thereof containing an antigenic determinant of the biomarker. Antibodies that can be used in the practice of the invention include, but are not limited to, monoclonal antibodies, polyclonal antibodies, chimeric antibodies, recombinant fragments of antibodies, Fab fragments, Fab′ fragments, F(ab′)₂ fragments, Fᵥ fragments, or scFᵥ fragments.

Methods of the invention, as described herein, can be used for determining the prognosis of a subject having dysplasia or cancer. The amount of PERP is measured in a biological sample derived from the subject, wherein complete loss of PERP indicates that the subject is at high risk of disease progression. In patients with dysplasia, loss of PERP is correlated with a high risk of progression to higher grades of dysplasia and invasive cancer. In patients with cancer, loss of PERP is correlated with a high risk of local relapse and more aggressive disease.

In another aspect, the invention includes a biomarker panel comprising PERP and one or more biomarkers for detecting desmosome deficiency (e.g., plakoglobin, desmoglein 1/3). In another embodiment, the invention includes a panel of biomarkers for diagnosis of cancer or dysplasia comprising PERP and further comprising one or more polypeptides selected from the group consisting of plakoglobin, desmoglein 1/3, E-cadherin, interleukin 1 family member 6 (IL1f6), S100a9, chitinase 3-like 1 (Chi311), chemokine ligand 20 (Ccl20), and interleukin-22 receptor (IL22ra).

In another embodiment, the invention includes a method for evaluating the effect of an agent for treating cancer or dysplasia in a subject, the method comprising: analyzing the amount of PERP in biological samples derived from the subject before and after the subject is treated with the agent, and comparing the amount of PERP with respective reference value ranges for PERP.

In another embodiment, the invention includes a method for monitoring the efficacy of a therapy for treating cancer or dysplasia in a subject, the method comprising: analyzing the amount of PERP in biological samples derived from the subject before and after the subject undergoes the therapy, and comparing the amount of PERP with respective reference value ranges for PERP.

In another aspect, the invention includes a kit for determining diagnosis or prognosis of cancer or dysplasia in a subject. The kit may include an agent for detecting PERP, a container for holding a biological sample isolated from a human subject suspected of having cancer or dysplasia; and printed instructions for reacting the agent with the biological sample or a portion of the biological sample to detect the presence or amount of PERP in the biological sample. The kit may further comprise agents for detecting one or more additional biomarkers for diagnosis or prognosis of cancer or dysplasia. The agents may be packaged in separate containers. The kit may further comprise one or more control reference samples and reagents for performing immunohistochemistry or other immunoassay. The kit may include antibodies that specifically bind to PERP. Additionally, the kit may include antibodies that specifically bind to biomarkers for desmosomes, such as plakoglobin and desmoglein 1/3, or antibodies that specifically bind to biomarkers for cancer or dysplasia, such as E-cadherin, interleukin 1 family member 6 (IL1f6), S100a9, chitinase 3-like 1 (Chi311), chemokine ligand 20 (Ccl20), and interleukin-22 receptor (IL22ra).

These and other embodiments of the subject invention will readily occur to those of skill in the art in view of the disclosure herein.

### BRIEF DESCRIPTION OF THE FIGURES

FIGS. 1A-1F show that Perp-deficiency promotes tumorigenesis. FIG. 1A shows Perp immunofluorescence images demonstrating the presence of Perp in the epidermis of control mice and the loss of Perp in the epidermis of K14CreER;Perp^{fl/fl} mice. Cells are shown with staining of Perp (light gray) or the desmosomal protein Desmoplakin (light gray), and with DAPI staining of nuclei (dark gray). Low magnification images show an overview of the epidermis (upper left), and high magnification images show the punctate staining pattern typical of desmosomal proteins (upper right). Punctate desmosomal pattern in the epidermis is comparable to that observed in wild-type mouse keratinocyte monolayers (lower panel). FIG. 1B shows the time line of the tumor study. FIG. 1C shows a Kaplan-Meier analysis of tumor latency in UVB-treated control (Perp^{fl/fl} and Perp^{fl/+}) and K14CreER;Perp^{fl/fl} mice. The statistical significance was determined using the Log Rank test (*p=0.0002, n=25 for each genotype). FIG. 1D shows a graph of the average number of SCCs per UVB-treated mouse ±STDEV (left). The statistical analysis was performed using the Student's unpaired t-test (*p=0.00049). Representative photographs of control and K14CreER;Perp^{fl/fl} mice (right) are shown with an arrow indicating the location of a tumor. FIG. 1E shows representative Hematoxylin and Eosin (H&E) stained images illustrating the various SCC grades. FIG. 1F shows a table indicating the numbers of SCCs of different grades in UVB-treated control and K14CreER;Perp^{fl/fl} mice.

FIGS. 2A-2G show that Perp loss compromises UVB-induced apoptosis in vivo and in vitro. FIG. 2A shows Perp immunohistochemistry demonstrating the loss of Perp in the epidermis of tamoxifen-treated K14CreER;Perp^{fl/fl} mice. The dashed line demarcates epidermis (EP) and dermis (D). FIG. 2B shows immunohistochemistry demonstrating p53 stabilization (arrows) in the epidermis of both control and K14CreER;Perp^{fl/fl} mice 24 hours after treatment with 2.5 kJ/m² UVB radiation. FIG. 2C shows cleaved Caspase 3 (CC3) immunohistochemistry, which was used to detect apoptosis in the epidermis of untreated and UVB-treated mice of different genotypes. The arrows indicate apoptotic cells. FIG. 2D shows quantification of apoptosis in untreated and UVB-treated control, K14CreER;Perp^{fl/fl}, and p53−/− mice. The graph depicts the average number of cleaved Caspase 3 (CC3)-positive cells per linear cm of epidermis, ±SEM. The data were derived from the analysis of segments of skin at least 2-3 cm long per mouse, in several independent experiments with the following numbers of mice: wild-type controls (n=8), K14CreER;Perp^{fl/fl} (n=12), p53−/− (n=5). Statistical analysis was conducted using the Student's unpaired t-test (*p=0.0017 versus treated wild-type and **p=0.0003 versus treated wild-type). FIG. 2E shows representative immunofluorescence images of wild-type, Perp−/−, and p53−/− keratinocyte monolayers, either untreated or treated with 1 kJ/m² UVB, and stained with a cleaved Caspase 3 antibody and DAPI to measure apoptosis. FIG. 2F shows higher magnification images of apoptotic cells, which display both cleaved Caspase 3-positivity and nuclear blebbing and chromatin condensation by DAPI staining, hallmarks of apoptosis. FIG. 2G shows quantitation of the percentage of apoptotic cells per 200× field in untreated and UVB-treated wild-type, Perp−/−, and p53−/− keratinocytes, as assessed by cleaved Caspase 3/DAPI staining. The graph represents the average ±SEM of three independent experiments performed in triplicate. The statistical analysis was conducted using the Student's unpaired t-test. (*p=0.011 versus treated wild-type and **p=0.0029 versus treated wild-type).

FIGS. 3A-3E show an adhesion junction analysis in tumors from K14CreER;Perp^{fl/fl} and control mice. FIG. 3A shows representative Hematoxylin and Eosin (H&E) staining and immunofluorescence images of desmosome and adherens junction component protein expression in skin from tamoxifen-treated K14CreER;Perp^{fl/fl} mice demonstrating that Perp loss itself does not disrupt membrane expression of other adhesion proteins. FIG. 3B shows a western blot of proteins from both the Triton X-100-soluble and urea-only soluble fractions of mouse epidermal lysates from control and K14CreER;Perp^{fl/fl} mice. Desmoglein 1/2 (Dsg 1/2) and Plakoglobin (Pg) solubility were examined. Gapdh serves as a loading control for the Triton X-100-soluble pool, whereas Keratin 14 serves as the loading control for the urea fraction. FIG. 3C shows representative H&E and immunofluorescence images of a K14CreER;Perp^{fl/fl} tumor sample demonstrating desmosomal component loss with retention of adherens junction components. Antigen staining is shown in light gray, and DAPI-marked nuclei are shown in dark gray. Tumors from K14CreER;Perp^{fl/fl} mice were stained with antibodies against various desmosomal and adherens junction components, and both the percentage of epithelial cells staining positive for each marker and the intensity of staining were measured. Staining for each antigen was categorized as high (>70%), medium (30-70%), or low (<30%) level expression. FIG. 3D shows a graph of the percentages of K14CreER; Perp^{fl/fl} tumors categorized into respective groups based on quantitative immunofluorescence staining for Plakoglobin and Desmoglein 1/3 (left); and a graph depicting the percentages of tumors categorized into respective groups based on immunofluorescence staining for E-cadherin and Beta-catenin (right). FIG. 3E shows tumors from control wild-type mice, stained for desmosomal and adherens junction components. Each component was categorized as displaying high, medium, or low level expression, as described above. The graph (left) shows the percentages of control tumors categorized into respective groups based on quantitative immunofluorescence staining for Perp, Plakoglobin, and Desmoglein 1/3. A second graph (right) depicts the percentages of tumors categorized into respective groups based on immunofluorescence staining for E-cadherin and Beta-catenin.

FIGS. 4A-4B show PERP loss with E-cadherin maintenance is a common event in human skin SCCs. FIG. 4A shows representative PERP and E-cadherin immunostaining of SCCs illustrating different expression patterns at low (left 100:1) and high (right 400:1) power magnification. Examples of PERP+;E-cadherin+ (i), PERP−;E-cadherin− (ii), and PERP−;E-cadherin+ (iii) tumors are shown. Dashed boxes indicate regions shown in the high magnification images. FIG. 4B shows a table quantifying the numbers and percentages of SCCs with specific staining patterns for PERP and E-cadherin expression. Note the high percentage of tumors exhibiting strong E-cadherin staining, but no PERP staining.

FIGS. 5A-5I show Perp-deficiency induces expression of inflammation-related genes. FIG. 5A shows Perp immunofluorescence demonstrating Perp loss in the epidermis of K14CreER;Perp^{fl/fl} mice two weeks after tamoxifen injection. FIG. 5B shows genes that are differentially expressed between control K14CreER;wild-type and K14CreER;Perp^{fl/fl} skin, which were identified using SAM (Significance Analysis of Microarrays) with an FDR of 10%. The 143 genes (51 induced and 92 repressed in Perp-deficient skin compared to control skin) are grouped by hierarchical clustering and represented in the heat map. FIG. 5C shows major classes of genes that are upregulated and downregulated in K14CreER;Perp^{fl/fl} skin compared to controls, as determined by Gene Ontology (GO) annotation. Members of the metabolic process, transport, immune system process, developmental process, and cell communication categories were statistically significantly enriched (p=6.7361024, p=3.5261023, p=8.9161023; p=1.5961022, p=2.4761022, respectively, by the binomial statistic). FIG. 5D shows a table of genes induced 3-fold or greater in K14CreER;Perp^{fl/fl} skin relative to control samples. FIG. 5E shows a quantitative-RT-PCR analysis validating Il1f6 (*p=3.161025), s100a9 (*p=0.00022), Chi311 (*p=1.261026), and Ccl20 (*p=0.0002) as genes induced upon Perp loss. Graphs represent the average expression levels in the skin of five mice examined in triplicate ±SEM. Statistical significance was calculated using the Student's unpaired t-test. FIG. 5F shows representative immunofluorescence images of CD3-positive T-cells in control versus K14CreER;Perp^{fl/fl} mouse skin. T-cells are stained in green (arrows) and nuclei are stained with DAPI in blue. EP indicates epidermis and D indicates dermis, with white dashed line delineating the boundary between the two compartments. FIG. 5G shows quantification of CD3-positive T-cells in the skin of control and K14CreER;Perp^{fl/fl} mice. Graph represents the average number of CD3-positive T-cells counted in triplicate 200× fields, from the skin of each of at least 5 mice, ±SEM. Statistical significance was analyzed using the Student's unpaired t-test. (p=0.21). FIG. 5H shows representative images of staining for toluidine blue-positive mast cells in control and K14CreER;Perp^{fl/fl} mouse skin. Dashed box represents area seen in higher magnification (400:1) images below. Note that mast cells are identified by the purple stain (arrows), which differs from the blue stained background due to pH differences within mast cells. FIG. 5I shows quantification of mast cell numbers in the skin of control and K14CreER;Perp^{fl/fl} mice. The graph represents the average number of mast cells counted in triplicate 200× fields, from the skin of each of 5 mice, ±SEM. (p=0.7; Student's unpaired t-test).

FIGS. 6A-6F show that combined Perp-deficiency and chronic UVB exposure induce immune cell infiltration in the skin. FIG. 6A show representative immunofluorescence images of myeloid cells, as determined by MPO staining (arrows), in the skin of control and K14CreER;Perp^{fl/fl} mice treated with UVB light for 19 weeks. FIG. 6B shows the quantification of MPO-positive cells in UVB-treated cohorts. The graph represents the average of 3 mice, quantified in triplicate 200× fields ±SEM. (p=0.89; Student's unpaired t-test). FIG. 6C shows representative immunofluorescence images of T-cells, as assessed by CD3 staining (arrows), in the skin of control and K14CreER;Perp^{fl/fl} mice treated with UVB light for 19 weeks. FIG. 6D shows the quantification of CD3-positive T-cell numbers in UVB-treated cohorts. Graph represents the average number of T-cells in the skin of 3 mice, quantified in triplicate 200× fields, ±SEM (*p=0.044, Student's unpaired t-test). FIG. 6E shows representative images of staining for mast cells, as assessed by toluidine blue-positivity (arrows), in the skin of control and K14CreER;Perp^{fl/fl} mice treated with UVB light for 19 weeks. Note the increase in mast cells underlying the epidermis in the K14CreER;Perp^{fl/fl} mice. FIG. 6F shows the quantification of mast cells in UVB-treated cohorts. The graph represents the average of 3 mice, quantified in triplicate 200× fields ±SEM (*p=0.0092; Student's unpaired t-test).

FIGS. 7A-7C show a model for how Perp-deficiency can promote tumorigenesis. Perp loss, combined with chronic UVB exposure, can promote cancer through three mechanisms. FIG. 7A shows that compromised apoptosis in the epidermis of Perp-deficient mice in response to UVB light can lead to inappropriate survival of cells sustaining DNA damage and expansion of pre-malignant cells. FIG. 7B shows that impaired desmosomal adhesion in Perp-deficient mice, depicted by downregulation of a desmosomal cadherin, can facilitate the complete disruption of desmosomes that stimulates tumorigenesis. The exact placement of Perp, a tetraspan membrane protein, within the desmosome is speculative. FIG. 7C shows that the recruitment of inflammatory cells to the skin of UVB treated Perp-deficient mice can promote cancer through mechanisms such as enhancing remodeling of the tumor microenvironment or stimulating angiogenesis.

FIGS. 8A-8G show representative cores from TMA1: Normal mucosa (FIG. 8A) with full-thickness membrane staining with PERP (Fig. B) contrasts with the loss of membrane staining (FIGS. 8D, 8F, 8H) seen with carcinoma in situ (CIS) (FIG. 8C), invasive cancer (FIG. 8E) and cervical nodal metastasis (FIG. 8G). Cytoplasmic only staining is non-specific and scored as negative.

FIG. 9 shows the distribution of extent of PERP membrane staining for normal, CIS, invasive carcinoma, and metastasis (TMA1).

FIGS. 10A-10F show representative cores from TMA2: Mild dysplasia (FIG. 10A) with full-thickness membrane staining with PERP (FIG. 10B) contrasts with moderate dysplasia (FIG. 10C) showing partial loss of PERP (FIG. 10D) and severe dysplasia (FIG. 10E) showing complete loss of PERP (FIG. 10F). Cytoplasmic only staining is nonspecific and scored as negative.

FIG. 11 shows the distribution of extent of PERP membrane staining for mild, moderate, and severe dysplasia (TMA2).

FIG. 12 shows a graph depicting freedom from local relapse by extent of PERP loss within the invasive component.

### DETAILED DESCRIPTION

The practice of the present invention will employ, unless otherwise indicated, conventional methods of pharmacology, chemistry, biochemistry, recombinant DNA techniques and immunology, within the skill of the art. Such techniques are explained fully in the literature. See, e.g., Handbook of Experimental Immunology, Vols. I-IV (D. M. Weir and C. C. Blackwell eds., Blackwell Scientific Publications); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.).

All publications, patents and patent applications cited herein, whether supra or infra, are hereby incorporated by reference in their entireties.

### I. DEFINITIONS

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “a biomarker” includes a mixture of two or more biomarkers, and the like.

The term “about”, particularly in reference to a given quantity, is meant to encompass deviations of plus or minus five percent.

A “biomarker” in the context of the present invention refers to a polypeptide which is differentially expressed in a sample taken from patients having dysplasia or cancer as compared to a comparable sample taken from control subjects (e.g., a person with a negative diagnosis, normal or healthy subject). Biomarkers include, but are not limited to, the p53 apoptosis effector related to PMP-22 (PERP), plakoglobin, desmoglein 1/3, E-cadherin, interleukin 1 family member 6 (IL1f6), S100a9, chitinase 3-like 1 (Chi311), chemokine ligand 20 (Ccl20), and interleukin-22 receptor (IL22ra).

The terms “tumor,” “cancer” and “neoplasia” are used interchangeably and refer to a cell or population of cells whose growth, proliferation or survival is greater than growth, proliferation or survival of a normal counterpart cell, e.g. a cell proliferative, hyperproliferative or differentiative disorder. Typically, the growth is uncontrolled. The term “malignancy” refers to invasion of nearby tissue. The term “metastasis” or a secondary, recurring or recurrent tumor, cancer or neoplasia refers to spread or dissemination of a tumor, cancer or neoplasia to other sites, locations or regions within the subject, in which the sites, locations or regions are distinct from the primary tumor or cancer. Neoplasia, tumors and cancers include benign, malignant, metastatic and non-metastatic types, and include any stage (I, II, III, IV or V) or grade (G1, G2, G3, etc.) of neoplasia, tumor, or cancer, or a neoplasia, tumor, cancer or metastasis that is progressing, worsening, stabilized or in remission. In particular, the terms “tumor,” “cancer” and “neoplasia” include carcinomas, such as squamous cell carcinoma, adenocarcinoma, adenosquamous carcinoma, anaplastic carcinoma, large cell carcinoma, and small cell carcinoma. These terms include, but are not limited to, skin cancer, head cancer, neck cancer, oral cavity cancer, tongue cancer, throat cancer, breast cancer, prostate cancer, lung cancer, ovarian cancer, testicular cancer, colon cancer, pancreatic cancer, and brain cancer.

The phrase “differentially expressed” refers to differences in the quantity and/or the frequency of a biomarker present in a sample taken from patients having, for example, dysplasia or cancer as compared to a control subject. For example, a biomarker can be a polypeptide which is present at an elevated level or at a decreased level in samples of patients with dysplasia or cancer compared to samples of control subjects. Alternatively, a biomarker can be a polypeptide which is detected at a higher frequency or at a lower frequency in samples of patients compared to samples of control subjects. A biomarker can be differentially present in terms of quantity, frequency or both.

A polypeptide is differentially expressed between two samples if the amount of the polypeptide in one sample is statistically significantly different from the amount of the polypeptide in the other sample. For example, a polypeptide is differentially expressed in two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

Alternatively or additionally, a polypeptide is differentially expressed in two sets of samples if the frequency of detecting the polypeptide in samples of patients suffering from dysplasia or cancer, is statistically significantly higher or lower than in the control samples. For example, a polypeptide is differentially expressed in two sets of samples if it is detected at least about 120%, at least about 130%, at least about 150%, at least about 180%, at least about 200%, at least about 300%, at least about 500%, at least about 700%, at least about 900%, or at least about 1000% more frequently or less frequently observed in one set of samples than the other set of samples.

The terms “subject,” “individual,” and “patient,” are used interchangeably herein and refer to any mammalian subject for whom diagnosis, prognosis, treatment, or therapy is desired, particularly humans. Other subjects may include cattle, dogs, cats, guinea pigs, rabbits, rats, mice, horses, and so on. In some cases, the methods of the invention find use in experimental animals, in veterinary application, and in the development of animal models for disease, including, but not limited to, rodents including mice, rats, and hamsters; and primates.

As used herein, a “biological sample” refers to a sample of tissue or cells isolated from a subject, including but not limited to, samples of skin, organs, biopsies and also samples from in vitro cell culture resulting from the growth of cells or tissues in culture medium, including recombinant cells and cell components.

The terms “quantity,” “amount,” and “level” are used interchangeably herein and may refer to an absolute quantification of a molecule or an analyte in a sample, or to a relative quantification of a molecule or analyte in a sample, i.e., relative to another value such as relative to a reference value as taught herein, or to a range of values for the biomarker. These values or ranges can be obtained from a single patient or from a group of patients.

A “test amount” of a biomarker refers to an amount of a biomarker present in a sample being tested. A test amount can be either an absolute amount (e.g., μg/ml) or a relative amount (e.g., relative intensity of signals).

A “diagnostic amount” of a biomarker refers to an amount of a biomarker in a subject's sample that is consistent with a diagnosis of dysplasia or cancer. A diagnostic amount can be either an absolute amount (e.g., μg/ml) or a relative amount (e.g., relative intensity of signals).

A “control amount” of a marker can be any amount or a range of amount which is to be compared against a test amount of a marker. For example, a control amount of a biomarker can be the amount of a biomarker in a person without dysplasia or cancer. A control amount can be either in absolute amount (e.g., μg/ml) or a relative amount (e.g., relative intensity of signals).

The term “antibody” encompasses polyclonal and monoclonal antibody preparations, as well as preparations including hybrid antibodies, altered antibodies, chimeric antibodies and, humanized antibodies, as well as: hybrid (chimeric) antibody molecules (see, for example, Winter et al. (1991) Nature 349:293-299; and U.S. Pat. No. 4,816,567); F(ab′)₂ and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, e.g., Huston et al. (1988) Proc Natl Acad Sci USA 85:5879-5883); dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

“Immunoassay” is an assay that uses an antibody to specifically bind an antigen (e.g., a biomarker). The immunoassay is characterized by the use of specific binding properties of a particular antibody to isolate, target, and/or quantify the antigen. An immunoassay for a biomarker may utilize one antibody or several antibodies. Immunoassay protocols may be based, for example, upon competition, direct reaction, or sandwich type assays using, for example, labeled antibody. The labels may be, for example, fluorescent, chemiluminescent, or radioactive.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to a biomarker from specific species such as rat, mouse, or human can be selected to obtain only those polyclonal antibodies that are specifically immunoreactive with the biomarker and not with other proteins, except for polymorphic variants and alleles of the biomarker. This selection may be achieved by subtracting out antibodies that cross-react with biomarker molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane. Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

“Capture reagent” refers to a molecule or group of molecules that specifically bind to a specific target molecule or group of target molecules. For example, a capture reagent can comprise two or more antibodies each antibody having specificity for a separate target molecule. Capture reagents can be any combination of organic or inorganic chemicals, or biomolecules, and all fragments, analogs, homologs, conjugates, and derivatives thereof that can specifically bind a target molecule.

The capture reagent can comprise a single molecule that can form a complex with multiple targets, for example, a multimeric fusion protein with multiple binding sites for different targets. The capture reagent can comprise multiple molecules each having specificity for a different target, thereby resulting in multiple capture reagent-target complexes. In certain embodiments, the capture reagent is comprised of proteins, such as antibodies.

The capture reagent can be directly labeled with a detectable moiety. For example, an anti-biomarker antibody can be directly conjugated to a detectable moiety and used in the inventive methods, devices, and kits. In the alternative, detection of the capture reagent-biomarker complex can be by a secondary reagent that specifically binds to the biomarker or the capture reagent-biomarker complex. The secondary reagent can be any biomolecule, and is preferably an antibody. The secondary reagent is labeled with a detectable moiety. In some embodiments, the capture reagent or secondary reagent is coupled to biotin, and contacted with avidin or streptavidin having a detectable moiety tag.

“Detectable moieties” or “detectable labels” contemplated for use in the invention include, but are not limited to, radioisotopes, fluorescent dyes such as fluorescein, phycoerythrin, Cy-3, Cy-5, allophycoyanin, DAPI, Texas Red, rhodamine, Oregon green, Lucifer yellow, and the like, green fluorescent protein (GFP), red fluorescent protein (DsRed), Cyan Fluorescent Protein (CFP), Yellow Fluorescent Protein (YFP), Cerianthus Orange Fluorescent Protein (cOFP), alkaline phosphatase (AP), beta-lactamase, chloramphenicol acetyltransferase (CAT), adenosine deaminase (ADA), aminoglycoside phosphotransferase (neo^{r}, G418^{r}) dihydrofolate reductase (DHFR), hygromycin-B-phosphotransferase (HPH), thymidine kinase (TK), lacZ (encoding α-galactosidase), and xanthine guanine phosphoribosyltransferase (XGPRT), Beta-Glucuronidase (gus), Placental Alkaline Phosphatase (PLAP), Secreted Embryonic Alkaline Phosphatase (SEAP), or Firefly or Bacterial Luciferase (LUC). Enzyme tags are used with their cognate substrate. The terms also include color-coded microspheres of known fluorescent light intensities (see e.g., microspheres with xMAP technology produced by Luminex (Austin, Tex.); microspheres containing quantum dot nanocrystals, for example, containing different ratios and combinations of quantum dot colors (e.g., Qdot nanocrystals produced by Life Technologies (Carlsbad, Calif.); glass coated metal nanoparticles (see e.g., SERS nanotags produced by Nanoplex Technologies, Inc. (Mountain View, Calif.); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), and glass microparticles with digital holographic code images (see e.g., CyVera microbeads produced by Illumina (San Diego, Calif.). As with many of the standard procedures associated with the practice of the invention, skilled artisans will be aware of additional labels that can be used.

“Diagnosis” as used herein generally includes determination as to whether a subject is likely affected by a given disease, disorder or dysfunction. The skilled artisan often makes a diagnosis on the basis of one or more diagnostic indicators, i.e., a biomarker, the presence, absence, or amount of which is indicative of the presence or absence of the disease, disorder or dysfunction.

“Prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

“Substantially purified” refers to nucleic acid molecules or proteins that are removed from their natural environment and are isolated or separated, and are at least about 60% free, preferably about 75% free, and most preferably about 90% free, from other components with which they are naturally associated.

### II. MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention is based on the discovery that PERP can be used as a biomarker for diagnosis of cancer and dysplasia. In particular, the inventors have discovered that PERP deficiency is associated with the progression of low grade dysplasia to higher grades of dysplasia and to cancer (see Examples 1 and 2). The inventors have further shown that PERP is useful for identifying patients with increased risk of progression to higher grade disease and to invasive carcinoma (see Example 2). In order to further an understanding of the invention, a more detailed discussion is provided below regarding PERP as a biomarker and methods of using PERP in prognosis, diagnosis, and monitoring treatment of cancer and dysplasia.

A. Biomarkers

PERP is useful as a prognostic or diagnostic marker and for monitoring treatment of dysplasia and cancer. It can be used alone or in combination with one or more additional biomarkers or relevant clinical parameters in prognosis, diagnosis, or monitoring treatment of cancer or dysplasia. In one embodiment, PERP is used in combination with one or more biomarkers for desmosomes, such as plakoglobin and desmoglein 1/3 for detection of desmosome deficiency. In another embodiment, PERP is used in combination with one or more biomarkers for cancer or dysplasia, such as E-cadherin, interleukin 1 family member 6 (IL1f6), S100a9, chitinase 3-like 1 (Chi311), chemokine ligand 20 (Ccl20), and interleukin-22 receptor (IL22ra). Expression profiles for PERP and these other biomarkers are useful for diagnosing dysplasia or cancer and for assessing the risk of disease progression and relapse.

Accordingly, in one aspect, the invention provides a method for diagnosing cancer or dysplasia in a subject, comprising measuring the amount of PERP in a biological sample derived from a subject suspected of having cancer or dysplasia, and analyzing the amount of PERP and comparing with reference value ranges for PERP. Decreased expression of PERP in a biological sample compared to the amount of PERP in a control sample indicates that a subject has cancer or dysplasia. When analyzing the levels of PERP in a biological sample, the reference value ranges used for comparison can represent the amount of PERP found in one or more samples of one or more subjects without cancer or dysplasia (i.e., normal or control samples). Alternatively, the reference value ranges can represent the amount of PERP found in one or more samples of one or more subjects with cancer or dysplasia. More specifically, the reference value ranges can represent the amount of PERP at particular stages of disease (e.g., mild, moderate, or severe dysplasia, cancer in situ, or invasive cancer) to facilitate a determination of the stage of disease progression in an individual.

The biological sample obtained from the subject to be diagnosed is typically a biopsy of abnormal tissue suspected of containing cancerous or dysplastic cells, but can be any sample of tissue or cells that contains desmosomes or PERP. The biological sample may include samples from in vitro cell culture resulting from the growth of cells or tissues from the subject in culture. The biological sample can be obtained from the subject by conventional techniques. For example, samples of tissue or cells can be obtained by surgical techniques well known in the art.

In certain embodiments, the biological sample may comprise a tissue sample including a portion, piece, part, segment, or fraction of a tissue which is obtained or removed from an intact tissue of a subject. Tissue samples can be obtained, for example, from the skin, oral cavity, tongue, head, neck, throat, breast, pancreas, stomach, liver, secretory gland, bladder, lung, prostate gland, ovary, cervix, uterus, brain, eye, connective tissue, bone, muscles or vasculature. A tissue biopsy may be obtained by methods including, but not limited to, an aspiration biopsy, a brush biopsy, a surface biopsy, a needle biopsy, a punch biopsy, an excision biopsy, an open biopsy, an incision biopsy or an endoscopic biopsy.

In certain embodiments, the biological sample is a tumor sample, including the entire tumor or a portion, piece, part, segment, or fraction of a tumor. A tumor sample can be obtained from a solid tumor or from a non-solid tumor, for example, from a squamous cell carcinoma, skin carcinoma, oral cavity carcinoma, head carcinoma, throat carcinoma, neck carcinoma, breast carcinoma, lung carcinoma, basal cell carcinoma, a colon carcinoma, a cervical carcinoma, Kaposi sarcoma, prostate carcinoma, an adenocarcinoma, a melanoma, hemangioma, meningioma, astrocytoma, neuroblastoma, carcinoma of the pancreas, gastric carcinoma, colorectal carcinoma, colon carcinoma, transitional cell carcinoma of the bladder, carcinoma of the larynx, chronic myeloid leukemia, acute lymphocytic leukemia, acute promyelocytic leukemia, multiple myeloma, T-cell lymphoma, B-cell lymphomas, retinoblastoma, sarcoma gallbladder, or bronchial cancer. The tumor sample may be obtained from a primary tumor or from a metastatic lesion.

A “control” sample as used herein refers to a biological sample, such as tissue or cells that are not diseased. That is, a control sample is obtained from a normal subject (e.g. an individual known to not have cancer or dysplasia or any condition or symptom associated with abnormal cell maturation or proliferation).

In one embodiment, the invention includes a biomarker panel comprising PERP and one or more other biomarkers for diagnosing cancer or dysplasia, wherein the biomarkers are selected from the group consisting of plakoglobin, desmoglein 1/3, E-cadherin, interleukin 1 family member 6 (IL1f6), S100a9, chitinase 3-like 1 (Chi311), chemokine ligand 20 (Ccl20), and interleukin-22 receptor (IL22ra). In one embodiment, the biomarker panel comprises PERP, plakoglobin, and desmoglein 1/3. In another embodiment, the biomarker panel comprises PERP, IL1f6, S100a9, Chi311, and Ccl20. In another embodiment, the biomarker panel comprises plakoglobin, desmoglein 1/3, E-cadherin, IL1f6, S100a9, Chi311, Ccl20, and IL22ra. Such biomarker panels can be used for diagnosis of dysplasia or cancer in a subject.

Biomarker panels of any size can be used in the practice of the invention, but typically comprise at least 3 biomarkers and up to 30 biomarkers, including any number of biomarkers in between, such as 3, 4, 5, 6, 7, 8, 9, 10, 11, 12, 13, 14, 15, 16, 17, 18, 19, 20, 21, 22, 23, 24, 25, 26, 27, 28, 29, or 30 biomarkers. For example, the biomarker panel may comprise 2-4 biomarkers, 5-7 biomarkers, 8-10 biomarkers, 10-15, biomarkers, 15-20 biomarkers, 20-25 biomarkers, or 25-30 biomarkers. In certain embodiments, the invention includes a biomarker panel comprising at least 3, or at least 4, or at least 5, or at least 6, or at least 7, or at least 8, or at least 9, or at least 10 or more biomarkers. Although smaller biomarker panels are usually more economical, larger biomarker panels (i.e., greater than 30 biomarkers) have the advantage of providing more detailed information and can also be used in the practice of the invention.

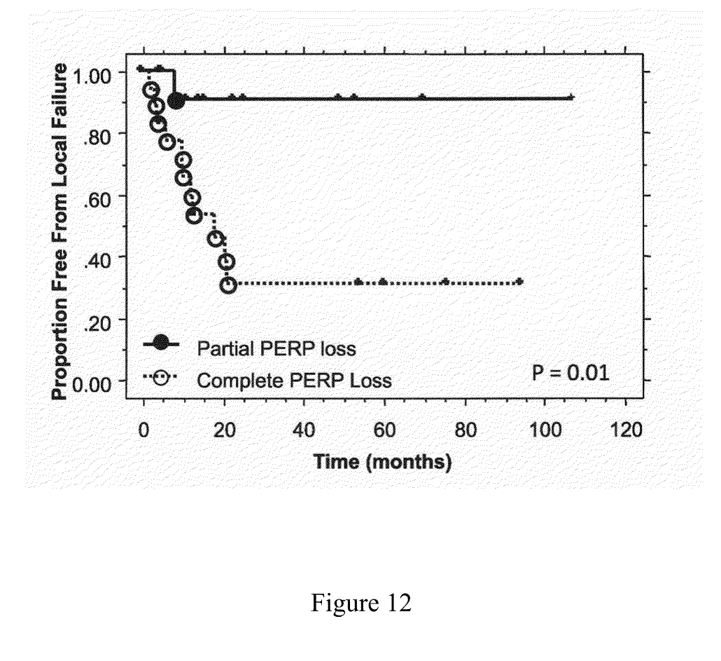

The methods of the invention, as described herein, can also be used for determining the prognosis of a subject and for monitoring treatment of a subject having dysplasia or cancer. The inventors have shown that loss of PERP is correlated with the severity of dysplasia or cancer and the likelihood of disease progression and relapse (see, e.g., Examples 1 and 2, Tables 2 and 3, and FIG. 12). Complete loss of PERP, as determined from analysis of a biological sample (e.g., a biopsy comprising dysplastic or cancerous cells or tissue), indicates that a subject is at high risk of disease progression. In patients with dysplasia, loss of PERP is correlated with a high risk of progression to higher grades of dysplasia and invasive cancer. In patients with cancer, loss of PERP is correlated with a high risk of local relapse and more aggressive disease.

Thus, a medical practitioner can monitor the progress of disease by measuring the level of PERP in a biological sample from the patient. For example, an increase in PERP level as compared to a prior PERP level (e.g., in a prior biological sample from the same area of lesion) indicates the disease or condition in the subject is improving or has improved, while a decrease of the PERP level as compared to a prior PERP level (e.g., in a prior biological sample from the same area of lesion) indicates the disease or condition in the subject has worsened or is worsening. Such worsening could possibly result in the recurrence of cancer or dysplasia.

The methods described herein for prognosis or diagnosis of cancer or dysplasia may be used in individuals who have not yet been diagnosed (for example, preventative screening), or who have been diagnosed, or who are suspected of having cancer or dysplasia (e.g., display one or more characteristic symptoms), or who are at risk of developing cancer or dysplasia (e.g., have a genetic predisposition or presence of one or more developmental, environmental, or behavioral risk factors). The methods may also be used to detect various stages of progression or severity of disease. The methods may also be used to detect the response of disease to prophylactic or therapeutic treatments or other interventions. The methods can furthermore be used to help the medical practitioner in determining prognosis (e.g., worsening, status-quo, partial recovery, or complete recovery) of the patient, and the appropriate course of action, resulting in either further treatment or observation, or in discharge of the patient from the medical care center.

B. Detecting and Measuring Levels of PERP and other Biomarkers

It is understood that the expression levels of PERP and other biomarkers in a sample can be determined by any suitable method known in the art. In one embodiment, the expression levels of the biomarkers are determined by measuring polypeptide levels of the biomarkers. Assays based on the use of antibodies that specifically recognize the proteins or polypeptide fragments of the biomarkers may be used for the measurement.

Such assays include, but are not limited to, immunohistochemistry (IHC), flow cytometry, cytometry by time of flight (CyTOF), western blotting, enzyme-linked immunosorbent assay (ELISA), radioimmunoassays (RIA), “sandwich” immunoassays, fluorescent immunoassays, enzyme multiplied immunoassay technique (EMIT), capillary electrophoresis immunoassays (CEIA) immunoprecipitation assays, the procedures of which are well known in the art (see, e.g., Ausubel et al, eds, 1994, Current Protocols in Molecular Biology, Vol. 1, John Wiley & Sons, Inc., New York, which is incorporated by reference herein in its entirety).

Antibodies that specifically bind to a biomarker can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986); and Kohler & Milstein, Nature 256:495-497 (1975). A biomarker antigen can be used to immunize a mammal, such as a mouse, rat, rabbit, guinea pig, monkey, or human, to produce polyclonal antibodies. If desired, a biomarker antigen can be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund's adjuvant, mineral gels (e.g., aluminum hydroxide), and surface active substances (e.g. lysolecithin, pluronic polyols, polyanions, peptides, oil emulsions, keyhole limpet hemocyanin, and dinitrophenol). Among adjuvants used in humans, BCG (bacilli Calmette-Guerin) and Corynebacterium parvum are especially useful.

Monoclonal antibodies which specifically bind to a biomarker antigen can be prepared using any technique which provides for the production of antibody molecules by continuous cell lines in culture. These techniques include, but are not limited to, the hybridoma technique, the human B cell hybridoma technique, and the EBV hybridoma technique (Kohler et al., Nature 256, 495-97, 1985; Kozbor et al., J. Immunol. Methods 81, 3142, 1985; Cote et al., Proc. Natl. Acad. Sci. 80, 2026-30, 1983; Cole et al., Mol. Cell Biol. 62, 109-20, 1984).

In addition, techniques developed for the production of “chimeric antibodies,” the splicing of mouse antibody genes to human antibody genes to obtain a molecule with appropriate antigen specificity and biological activity, can be used (Morrison et al., Proc. Natl. Acad. Sci. 81, 6851-55, 1984; Neuberger et al., Nature 312, 604-08, 1984; Takeda et al., Nature 314, 452-54, 1985). Monoclonal and other antibodies also can be “humanized” to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences can be minimized by replacing residues which differ from those in the human sequences by site directed mutagenesis of individual residues or by grating of entire complementarity determining regions.

Alternatively, humanized antibodies can be produced using recombinant methods, as described below. Antibodies which specifically bind to a particular antigen can contain antigen binding sites which are either partially or fully humanized, as disclosed in U.S. Pat. No. 5,565,332. Human monoclonal antibodies can be prepared in vitro as described in Simmons et al., PLoS Medicine 4(5), 928-36, 2007.

Alternatively, techniques described for the production of single chain antibodies can be adapted using methods known in the art to produce single chain antibodies which specifically bind to a particular antigen. Antibodies with related specificity, but of distinct idiotypic composition, can be generated by chain shuffling from random combinatorial immunoglobin libraries (Burton, Proc. Natl. Acad. Sci. 88, 11120-23, 1991).

Single-chain antibodies also can be constructed using a DNA amplification method, such as PCR, using hybridoma cDNA as a template (Thirion et al., Eur. J. Cancer Prev. 5, 507-11, 1996). Single-chain antibodies can be mono- or bispecific, and can be bivalent or tetravalent. Construction of tetravalent, bispecific single-chain antibodies is taught, for example, in Coloma & Morrison, Nat. Biotechnol. 15, 159-63, 1997. Construction of bivalent, bispecific single-chain antibodies is taught in Mallender & Voss, J. Biol. Chem. 269, 199-206, 1994.

A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology (Verhaar et al., Int. J Cancer 61, 497-501, 1995; Nicholls et al., J. Immunol. Meth. 165, 81-91, 1993).

Antibodies which specifically bind to a biomarker antigen also can be produced by inducing in vivo production in the lymphocyte population or by screening immunoglobulin libraries or panels of highly specific binding reagents as disclosed in the literature (Orlandi et al., Proc. Natl. Acad. Sci. 86, 3833 3837, 1989; Winter et al., Nature 349, 293 299, 1991).

Chimeric antibodies can be constructed as disclosed in WO 93/03151. Binding proteins which are derived from immunoglobulins and which are multivalent and multispecific, such as the “diabodies” described in WO 94/13804, also can be prepared.

Antibodies can be purified by methods well known in the art. For example, antibodies can be affinity purified by passage over a column to which the relevant antigen is bound. The bound antibodies can then be eluted from the column using a buffer with a high salt concentration.

Antibodies may be used in diagnostic assays to detect the presence or for quantification of the biomarkers in a biological sample. Such a diagnostic assay may comprise at least two steps; (i) contacting a biological sample with the antibody, wherein the sample is a tissue (e.g., human, animal, etc.), biological fluid (e.g., blood, urine, sputum, semen, amniotic fluid, saliva, etc.), biological extract (e.g., tissue or cellular homogenate, etc.), a protein microchip (e.g., See Arenkov P, et al., Anal Biochem., 278(2):123-131 (2000)), or a chromatography column, etc; and (ii) quantifying the antibody bound to the substrate. The method may additionally involve a preliminary step of attaching the antibody, either covalently, electrostatically, or reversibly, to a solid support, before subjecting the bound antibody to the sample, as defined above and elsewhere herein.

Various diagnostic assay techniques are known in the art, such as competitive binding assays, direct or indirect sandwich assays and immunoprecipitation assays conducted in either heterogeneous or homogenous phases (Zola, Monoclonal Antibodies: A Manual of Techniques, CRC Press, Inc., (1987), pp 147-158). The antibodies used in the diagnostic assays can be labeled with a detectable moiety. The detectable moiety should be capable of producing, either directly or indirectly, a detectable signal. For example, the detectable moiety may be a radioisotope, such as ²H, ¹⁴C, ³²P, or ¹²⁵I, a florescent or chemiluminescent compound, such as fluorescein isothiocyanate, rhodamine, or luciferin, or an enzyme, such as alkaline phosphatase, beta-galactosidase, green fluorescent protein, or horseradish peroxidase. Any method known in the art for conjugating the antibody to the detectable moiety may be employed, including those methods described by Hunter et al., Nature, 144:945 (1962); David et al., Biochem., 13:1014 (1974); Pain et al., J. Immunol. Methods, 40:219 (1981); and Nygren, J. Histochem. and Cytochem., 30:407 (1982).

Immunoassays can be used to determine the presence or absence of a biomarker in a sample as well as the quantity of a biomarker in a sample. First, a test amount of a biomarker in a sample can be detected using the immunoassay methods described above. If a biomarker is present in the sample, it will form an antibody-biomarker complex with an antibody that specifically binds the biomarker under suitable incubation conditions, as described above. The amount of an antibody-biomarker complex can be determined by comparing to a standard. A standard can be, e.g., a known compound or another protein known to be present in a sample. As noted above, the test amount of a biomarker need not be measured in absolute units, as long as the unit of measurement can be compared to a control.

Immunohistochemistry can be used to detect biomarker antigens in cells of a tissue section. For example, immunohistochemical staining with labeled antibodies can be used to detect abnormal cells, such as dysplastic or cancerous cells. Antibodies conjugated to enzymes, which catalyze color-producing reactions with chromogenic, fluorogenic, or chemiluminescent substrates (e.g., alkaline phosphatase or peroxidase), are commonly used. Alternatively, immunohistochemical staining can be performed with antibodies conjugated to fluorophores (e.g., fluorescein or rhodamine) to visualize biomarkers. See, e.g., Dabbs Diagnostic Immunohistochemistry: Theranostic and Genomic Applications, Saunders, 3^{rd} edition, 2010; Chu Modern Immunohistochemistry (Cambridge Illustrated Surgical Pathology) Cambridge University Press, 2009; Buchwalow et al. Immunohistochemistry: Basics and Methods, Springer, 1st Edition, 2010; and Ramos-Vara (2011) Methods Mol. Biol. 691:83-96; herein incorporated by reference in their entireties.

Flow cytometry can be used to detect multiple surface and intracellular biomarkers simultaneously in whole cells and to distinguish populations of cells expressing different cellular biomarkers. Typically, whole cells are incubated with antibodies that specifically bind to the biomarkers. The antibodies can be labeled, for example, with a fluorophore, isotope, or quantum dot to facilitate detection of the biomarkers. The cells are then suspended in a stream of fluid and passed through an electronic detection apparatus. (See, e.g., Shapiro Practical Flow Cytometry, Wiley-Liss, 4^{th} edition, 2003; Loken Immunofluorescence Techniques in Flow Cytometry and Sorting, Wiley, 2^{nd} edition, 1990; Flow Cytometry: Principles and Applications, (ed. Macey), Humana Press 1^{st} edition, 2007; herein incorporated by reference in their entireties.)

It may be useful in the practice of the invention to fractionate biological samples, e.g., to enrich samples for lower abundance proteins to facilitate detection of biomarkers, or to partially purify biomarkers isolated from biological samples to generate specific antibodies to biomarkers. There are many ways to reduce the complexity of a sample based on the binding properties of the proteins in the sample, or the characteristics of the proteins in the sample.

In one embodiment, a sample can be fractionated according to the size of the proteins in a sample using size exclusion chromatography. For a biological sample wherein the amount of sample available is small, preferably a size selection spin column is used. In general, the first fraction that is eluted from the column (“fraction 1”) has the highest percentage of high molecular weight proteins; fraction 2 has a lower percentage of high molecular weight proteins; fraction 3 has even a lower percentage of high molecular weight proteins; fraction 4 has the lowest amount of large proteins; and so on. Each fraction can then be analyzed by immunoassays, gas phase ion spectrometry, and the like, for the detection of biomarkers.

In another embodiment, a sample can be fractionated by anion exchange chromatography. Anion exchange chromatography allows fractionation of the proteins in a sample roughly according to their charge characteristics. For example, a Q anion-exchange resin can be used (e.g., Q HyperD F, Biosepra), and a sample can be sequentially eluted with eluants having different pH's. Anion exchange chromatography allows separation of biomarkers in a sample that are more negatively charged from other types of biomarkers. Proteins that are eluted with an eluant having a high pH are likely to be weakly negatively charged, and proteins that are eluted with an eluant having a low pH are likely to be strongly negatively charged. Thus, in addition to reducing complexity of a sample, anion exchange chromatography separates proteins according to their binding characteristics.

In yet another embodiment, a sample can be fractionated by heparin chromatography. Heparin chromatography allows fractionation of the biomarkers in a sample also on the basis of affinity interaction with heparin and charge characteristics. Heparin, a sulfated mucopolysaccharide, will bind biomarkers with positively charged moieties, and a sample can be sequentially eluted with eluants having different pH's or salt concentrations. Biomarkers eluted with an eluant having a low pH are more likely to be weakly positively charged. Biomarkers eluted with an eluant having a high pH are more likely to be strongly positively charged. Thus, heparin chromatography also reduces the complexity of a sample and separates biomarkers according to their binding characteristics.

In yet another embodiment, a sample can be fractionated by isolating proteins that have a specific characteristic, e.g. glycosylation. For example, a CSF sample can be fractionated by passing the sample over a lectin chromatography column (which has a high affinity for sugars). Glycosylated proteins will bind to the lectin column and non-glycosylated proteins will pass through the flow through. Glycosylated proteins are then eluted from the lectin column with an eluant containing a sugar, e.g., N-acetyl-glucosamine and are available for further analysis.

In yet another embodiment, a sample can be fractionated using a sequential extraction protocol. In sequential extraction, a sample is exposed to a series of adsorbents to extract different types of biomarkers from a sample. For example, a sample is applied to a first adsorbent to extract certain proteins, and an eluant containing non-adsorbent proteins (i.e., proteins that did not bind to the first adsorbent) is collected. Then, the fraction is exposed to a second adsorbent. This further extracts various proteins from the fraction. This second fraction is then exposed to a third adsorbent, and so on.

Any suitable materials and methods can be used to perform sequential extraction of a sample. For example, a series of spin columns comprising different adsorbents can be used. In another example, a multi-well comprising different adsorbents at its bottom can be used. In another example, sequential extraction can be performed on a probe adapted for use in a gas phase ion spectrometer, wherein the probe surface comprises adsorbents for binding biomarkers. In this embodiment, the sample is applied to a first adsorbent on the probe, which is subsequently washed with an eluant. Biomarkers that do not bind to the first adsorbent are removed with an eluant. The biomarkers that are in the fraction can be applied to a second adsorbent on the probe, and so forth. The advantage of performing sequential extraction on a gas phase ion spectrometer probe is that biomarkers that bind to various adsorbents at every stage of the sequential extraction protocol can be analyzed directly using a gas phase ion spectrometer.

In yet another embodiment, biomarkers in a sample can be separated by high-resolution electrophoresis, e.g., one or two-dimensional gel electrophoresis. A fraction containing a biomarker can be isolated and further analyzed by gas phase ion spectrometry. Preferably, two-dimensional gel electrophoresis is used to generate a two-dimensional array of spots for the biomarkers. See, e.g., Jungblut and Thiede, Mass Spectr. Rev. 16:145-162 (1997).

Two-dimensional gel electrophoresis can be performed using methods known in the art. See, e.g., Deutscher ed., Methods In Enzymology vol. 182. Typically, biomarkers in a sample are separated by, e.g., isoelectric focusing, during which biomarkers in a sample are separated in a pH gradient until they reach a spot where their net charge is zero (i.e., isoelectric point). This first separation step results in one-dimensional array of biomarkers. The biomarkers in the one dimensional array are further separated using a technique generally distinct from that used in the first separation step. For example, in the second dimension, biomarkers separated by isoelectric focusing are further resolved using a polyacrylamide gel by electrophoresis in the presence of sodium dodecyl sulfate (SDS-PAGE). SDS-PAGE allows further separation based on molecular mass. Typically, two-dimensional gel electrophoresis can separate chemically different biomarkers with molecular masses in the range from 1000-200,000 Da, even within complex mixtures.

Biomarkers in the two-dimensional array can be detected using any suitable methods known in the art. For example, biomarkers in a gel can be labeled or stained (e.g., Coomassie Blue or silver staining). If gel electrophoresis generates spots that correspond to the molecular weight of one or more biomarkers of the invention, the spot can be further analyzed by densitometric analysis or gas phase ion spectrometry. For example, spots can be excised from the gel and analyzed by gas phase ion spectrometry. Alternatively, the gel containing biomarkers can be transferred to an inert membrane by applying an electric field. Then a spot on the membrane that approximately corresponds to the molecular weight of a biomarker can be analyzed by gas phase ion spectrometry. In gas phase ion spectrometry, the spots can be analyzed using any suitable techniques, such as MALDI or SELDI.

Prior to gas phase ion spectrometry analysis, it may be desirable to cleave biomarkers in the spot into smaller fragments using cleaving reagents, such as proteases (e.g., trypsin). The digestion of biomarkers into small fragments provides a mass fingerprint of the biomarkers in the spot, which can be used to determine the identity of the biomarkers if desired.

In yet another embodiment, high performance liquid chromatography (HPLC) can be used to separate a mixture of biomarkers in a sample based on their different physical properties, such as polarity, charge and size. HPLC instruments typically consist of a reservoir, the mobile phase, a pump, an injector, a separation column, and a detector. Biomarkers in a sample are separated by injecting an aliquot of the sample onto the column. Different biomarkers in the mixture pass through the column at different rates due to differences in their partitioning behavior between the mobile liquid phase and the stationary phase. A fraction that corresponds to the molecular weight and/or physical properties of one or more biomarkers can be collected. The fraction can then be analyzed by gas phase ion spectrometry to detect biomarkers.

Optionally, a biomarker can be modified before analysis to improve its resolution or to determine its identity. For example, the biomarkers may be subject to proteolytic digestion before analysis. Any protease can be used. Proteases, such as trypsin, that are likely to cleave the biomarkers into a discrete number of fragments are particularly useful. The fragments that result from digestion function as a fingerprint for the biomarkers, thereby enabling their detection indirectly. This is particularly useful where there are biomarkers with similar molecular masses that might be confused for the biomarker in question. Also, proteolytic fragmentation is useful for high molecular weight biomarkers because smaller biomarkers are more easily resolved by mass spectrometry. In another example, biomarkers can be modified to improve detection resolution. For instance, neuraminidase can be used to remove terminal sialic acid residues from glycoproteins to improve binding to an anionic adsorbent and to improve detection resolution. In another example, the biomarkers can be modified by the attachment of a tag of particular molecular weight that specifically binds to molecular biomarkers, further distinguishing them. Optionally, after detecting such modified biomarkers, the identity of the biomarkers can be further determined by matching the physical and chemical characteristics of the modified biomarkers in a protein database (e.g., SwissProt).

After preparation, biomarkers in a sample are typically captured on a substrate for detection. Traditional substrates include antibody-coated 96-well plates or nitrocellulose membranes that are subsequently probed for the presence of the proteins. Alternatively, protein-binding molecules attached to microspheres, microparticles, microbeads, beads, or other particles can be used for capture and detection of biomarkers. The protein-binding molecules may be antibodies, peptides, peptoids, aptamers, small molecule ligands or other protein-binding capture agents attached to the surface of particles. Each protein-binding molecule may comprise a “unique detectable label,” which is uniquely coded such that it may be distinguished from other detectable labels attached to other protein-binding molecules to allow detection of biomarkers in multiplex assays. Examples include, but are not limited to, color-coded microspheres with known fluorescent light intensities (see e.g., microspheres with xMAP technology produced by Luminex (Austin, Tex.); microspheres containing quantum dot nanocrystals, for example, having different ratios and combinations of quantum dot colors (e.g., Qdot nanocrystals produced by Life Technologies (Carlsbad, Calif.); glass coated metal nanoparticles (see e.g., SERS nanotags produced by Nanoplex Technologies, Inc. (Mountain View, Calif.); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), glass microparticles with digital holographic code images (see e.g., CyVera microbeads produced by Illumina (San Diego, Calif.); chemiluminescent dyes, combinations of dye compounds; and beads of detectably different sizes. See, e.g., U.S. Pat. No. 5,981,180, U.S. Pat. No. 7,445,844, U.S. Pat. No. 6,524,793, Rusling et al. (2010) Analyst 135(10): 2496-2511; Kingsmore (2006) Nat. Rev. Drug Discov. 5(4): 310-320, Proceedings Vol. 5705 Nanobiophotonics and Biomedical Applications II, Alexander N. Cartwright; Marek Osinski, Editors, pp. 114-122; Nanobiotechnology Protocols Methods in Molecular Biology, 2005, Volume 303; herein incorporated by reference in their entireties).

In another example, biochips can be used for capture and detection of proteins. Many protein biochips are described in the art. These include, for example, protein biochips produced by Packard BioScience Company (Meriden Conn.), Zyomyx (Hayward, Calif.) and Phylos (Lexington, Mass.). In general, protein biochips comprise a substrate having a surface. A capture reagent or adsorbent is attached to the surface of the substrate. Frequently, the surface comprises a plurality of addressable locations, each of which location has the capture reagent bound there. The capture reagent can be a biological molecule, such as a polypeptide or a nucleic acid, which captures other biomarkers in a specific manner. Alternatively, the capture reagent can be a chromatographic material, such as an anion exchange material or a hydrophilic material. Examples of such protein biochips are described in the following patents or patent applications: U.S. Pat. No. 6,225,047 (Hutchens and Yip, “Use of retentate chromatography to generate difference maps,” May 1, 2001), International publication WO 99/51773 (Kuimelis and Wagner, “Addressable protein arrays,” Oct. 14, 1999), International publication WO 00/04389 (Wagner et al., “Arrays of protein-capture agents and methods of use thereof,” Jul. 27, 2000), International publication WO 00/56934 (Englert et al., “Continuous porous matrix arrays,” Sep. 28, 2000).

In general, a sample containing the biomarkers is placed on the active surface of a biochip for a sufficient time to allow binding. Then, unbound molecules are washed from the surface using a suitable eluant. In general, the more stringent the eluant, the more tightly the proteins must be bound to be retained after the wash. The retained protein biomarkers now can be detected by any appropriate means, for example, mass spectrometry, fluorescence, surface plasmon resonance, ellipsometry or atomic force microscopy.

Mass spectrometry, and particularly SELDI mass spectrometry, is a particularly useful method for detection of the biomarkers of this invention. Laser desorption time-of-flight mass spectrometer can be used in embodiments of the invention. In laser desorption mass spectrometry, a substrate or a probe comprising biomarkers is introduced into an inlet system. The biomarkers are desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of markers of specific mass to charge ratio.

Matrix-assisted laser desorption/ionization mass spectrometry (MALDI-MS) can also be used for detecting the biomarkers of this invention. MALDI-MS is a method of mass spectrometry that involves the use of an energy absorbing molecule, frequently called a matrix, for desorbing proteins intact from a probe surface. MALDI is described, for example, in U.S. Pat. No. 5,118,937 (Hillenkamp et al.) and U.S. Pat. No. 5,045,694 (Beavis and Chait). In MALDI-MS, the sample is typically mixed with a matrix material and placed on the surface of an inert probe. Exemplary energy absorbing molecules include cinnamic acid derivatives, sinapinic acid (“SPA”), cyano hydroxy cinnamic acid (“CHCA”) and dihydroxybenzoic acid. Other suitable energy absorbing molecules are known to those skilled in this art. The matrix dries, forming crystals that encapsulate the analyte molecules. Then the analyte molecules are detected by laser desorption/ionization mass spectrometry.

Surface-enhanced laser desorption/ionization mass spectrometry, or SELDI-MS represents an improvement over MALDI for the fractionation and detection of biomolecules, such as proteins, in complex mixtures. SELDI is a method of mass spectrometry in which biomolecules, such as proteins, are captured on the surface of a protein biochip using capture reagents that are bound there. Typically, non-bound molecules are washed from the probe surface before interrogation. SELDI is described, for example, in: U.S. Pat. No. 5,719,060 (“Method and Apparatus for Desorption and Ionization of Analytes,” Hutchens and Yip, Feb. 17, 1998,) U.S. Pat. No. 6,225,047 (“Use of Retentate Chromatography to Generate Difference Maps,” Hutchens and Yip, May 1, 2001) and Weinberger et al., “Time-of-flight mass spectrometry,” in Encyclopedia of Analytical Chemistry, R. A. Meyers, ed., pp 11915-11918 John Wiley & Sons Chichesher, 2000.

Biomarkers on the substrate surface can be desorbed and ionized using gas phase ion spectrometry. Any suitable gas phase ion spectrometer can be used as long as it allows biomarkers on the substrate to be resolved. Preferably, gas phase ion spectrometers allow quantitation of biomarkers. In one embodiment, a gas phase ion spectrometer is a mass spectrometer. In a typical mass spectrometer, a substrate or a probe comprising biomarkers on its surface is introduced into an inlet system of the mass spectrometer. The biomarkers are then desorbed by a desorption source such as a laser, fast atom bombardment, high energy plasma, electrospray ionization, thermospray ionization, liquid secondary ion MS, field desorption, etc. The generated desorbed, volatilized species consist of preformed ions or neutrals which are ionized as a direct consequence of the desorption event. Generated ions are collected by an ion optic assembly, and then a mass analyzer disperses and analyzes the passing ions. The ions exiting the mass analyzer are detected by a detector. The detector then translates information of the detected ions into mass-to-charge ratios. Detection of the presence of biomarkers or other substances will typically involve detection of signal intensity. This, in turn, can reflect the quantity and character of biomarkers bound to the substrate. Any of the components of a mass spectrometer (e.g., a desorption source, a mass analyzer, a detector, etc.) can be combined with other suitable components described herein or others known in the art in embodiments of the invention.

The methods for detecting biomarkers in a sample have many applications. For example, PERP and one or more additional biomarkers can be measured to aid in the diagnosis or prognosis of cancer or dysplasia. In another example, the methods for detection of the biomarkers can be used to monitor responses in a subject to treatment. In another example, the methods for detecting biomarkers can be used to assay for and to identify compounds that modulate expression of these biomarkers in vivo or in vitro.

C. Kits

In yet another aspect, the invention provides kits for diagnosis or prognosis of cancer or dysplasia, wherein the kits can be used to detect PERP and optionally other biomarkers. For example, the kits can be used to detect PERP and additionally any one or more of the biomarkers described herein, which are differentially expressed in samples of patients having cancer or dysplasia and normal subjects. The kit may include one or more agents for detection of PERP and other biomarkers, a container for holding a biological sample isolated from a human subject suspected of having cancer or dysplasia; and printed instructions for reacting agents with the biological sample or a portion of the biological sample to detect the presence or amount of PERP and optionally other biomarkers in the biological sample. The agents may be packaged in separate containers. The kit may further comprise one or more control reference samples and reagents for performing an immunoassay.

In one embodiment, the kit comprises agents for measuring the levels of PERP and one or more other biomarkers of interest, including plakoglobin, desmoglein 1/3, E-cadherin, interleukin 1 family member 6 (IL1f6), S100a9, chitinase 3-like 1 (Chi311), chemokine ligand 20 (Ccl20), and interleukin-22 receptor (IL22ra). The kit may include antibodies that specifically bind to PERP. Additionally, the kit may include antibodies that specifically bind to biomarkers for desmosomes, such as plakoglobin and desmoglein 1/3, or antibodies that specifically bind to biomarkers for cancer or dysplasia, such as E-cadherin, interleukin 1 family member 6 (IL1f6), S100a9, chitinase 3-like 1 (Chi311), chemokine ligand 20 (Ccl20), and interleukin-22 receptor (IL22ra).

The kit can comprise one or more containers for compositions contained in the kit. Compositions can be in liquid form or can be lyophilized. Suitable containers for the compositions include, for example, bottles, vials, syringes, and test tubes. Containers can be formed from a variety of materials, including glass or plastic. The kit can also comprise a package insert containing written instructions for methods of diagnosing dysplasia or cancer.

The kits of the invention have a number of applications. For example, the kits can be used to determine if a subject has cancer or dysplasia. In another example, the kits can be used to determine the stage of cancer or dysplasia in a subject. In another example, the kits can be used to determine the prognosis (e.g., the likelihood of disease progression or relapse) for a subject having cancer or dysplasia. In another example, kits can be used to monitor the effectiveness of treatment of a patient having cancer or dysplasia. In a further example, the kits can be used to identify compounds that modulate expression of PERP or other biomarkers in in vitro or in vivo animal models to determine the effects of treatment.

### III. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### Identifying PERP as a Biomarker for Squamous Cell Carcinoma (SCC) in Mice

### Materials and Methods

### Tumor Study

All animal studies were approved by the Stanford University Administrative Panel on Laboratory Animal Care and were performed in strict accordance with IACUC guidelines. Keratin-14CreER^{T2} mice were bred to Perp^{fl/fl} conditional mice and kept on a 129/Sv; C57BL/6 mixed background (Metzger et al. (2005) Methods Mol Biol 289: 329-340). At 6 weeks of age, 0.1 mg of tamoxifen (Sigma Chemical Corp., St. Louis, Mo.) diluted first in ethanol then in corn oil was administered to mice for 5 consecutive days via intraperitoneal injection. Four weeks post-injection, mice began chronic UVB treatments (2.5 kJ/m², three times a week, for 30 weeks). Mice were shaved on a weekly basis and treated using Kodacel-filtered FS40 sunlamps. Mice were placed 5 in a cage and allowed to roam freely during treatment. Cages were rotated along the shelf below the light bulbs before each treatment to compensate for uneven distribution of energy along the bulbs. Mice were monitored for tumor incidence by visual inspection.

### Immunofluorescence/Immunohistochemistry

Tissue samples were fixed overnight in 10% formalin, processed, and embedded using standard procedures. Samples were deparaffinized, rehydrated, and unmasked using Trilogy (Cell Marque, Rocklin, Calif.) in a pressure cooker for 15 minutes according to the manufacturer's instructions. Samples were then rinsed in phosphate buffered saline (PBS) and blocked in PBS containing 5% normal goat serum (Sigma Chemical Corp.), 2.5% bovine serum albumin (Sigma Chemical Corp.), and 0.01% Triton X-100 (Fisher Scientific, Pittsburgh, Pa.). Sections were incubated in primary antibody overnight at 4° C., rinsed in PBS with 0.01% Tween-20 (Fisher Scientific), incubated with secondary antibody and 1 mg/mL 4′,6-diamidino-2-phenylindole (DAPI) (Sigma Chemical Corp.) for 1 hour at 37° C., and washed in PBS. Samples were mounted with Mowiol (EMD Chemicab, Gibbstown, N.J.). Fluorescence images were examined using a Leica DM6000B microscope (Leica Microsystems, Bannockburn, Ill.), and images were acquired using a Retiga Exi Camera (Q imaging, Surrey, British Columbia, Canada) and Image Pro 6.2 software from Media Cybernetics (Silver Spring, Md.).

### Keratinocyte Culture and Apoptosis Assays

Keratinocytes were derived from P.05-P1.5 mouse skin as described (Ihrie et al. (2005) Cell 120: 843-856). Cells were grown on collagen/fibronectin-coated dishes and maintained in an undifferentiated state by growing the cells in low calcium EMEM (Lonza, Basel, Switzerland) containing 0.05 mM calcium, 8% dialyzed FCS, and antibiotics. Cells were then differentiated for 24 hours in the same media as the undifferentiated cells, except the calcium concentration was raised to 2 mM. For immunofluorescence, keratinocytes were grown on collagen/fibronectin-coated glass coverslips. For UVB treatment, media was removed and cells were washed once in PBS, then treated with 1 kJ/m² UVB radiation using a Kodacel filter (Eastman Kodak, Rochester, N.Y.) to block residual UVC rays. After 48 hours, cells were fixed in cold methanol for 20 minutes at −20° C. Cells were stained with rabbit anti-cleaved Caspase 3 antibodies (Cell Signaling, Beverly, Mass.), followed by staining with FITC-anti-rabbit antibodies (Vector Laboratories, Burlingame, Calif.) and DAPI, and mounting using Mowiol.

### In Vivo Apoptosis Assays

Cohorts of K14CreER;Perp^{fl/fl} mice were generated, and at 6 weeks of age, mice were injected with tamoxifen, as described above. Four weeks later, the dorsal skins of mice were shaved. Mice were placed underneath a Kodacel filter and allowed to roam freely in their cage during UVB treatment. Half of the dorsal skin was exposed to a one time dose of 2.5 kJ/m² of UVB irradiation while the other half was blocked. 24 hours later the dorsal skins of the mice were collected and immunostained for cleaved Caspase 3. Apoptosis, indicated by cleaved Caspase 3-positivity, was quantified in at least 2-3 cm of skin per mouse.

### Antibodies

Primary antibodies against Perp (Ihrie et al., supra), Desmoglein 1 (Santa Cruz Biotechnology, Santa Cruz, Calif.), Plakoglobin (clone 11E4; Invitrogen), Keratin 14 (Covance, Princeton, N.J.), Alpha-tubulin (Sigma Chemical Corp.), E-cadherin (Invitrogen), GAPDH (Fitzgerald Industries, Acton, Mass.), Plakoglobin (1408; gift of K. Green), Smooth Muscle Actin (Santa Cruz Biotechnology), Desmoglein 1/3 (clone 32-2B; gift of D. Garrod), Desmoglein 1/2 (4B2; gift of K. Green), cleaved Caspase 3 (Cell Signaling), p53 (Vector Laboratories), Beta-catenin (BD Biosciences, San Jose, Calif.), Desmoplakin (11-5F; gift of D. Garrod), CD3 (Dako, Denmark), and MPO (AbCam, Cambridge, Mass.) were used in this study. The secondary antibodies that were used included FITC-anti-mouse, FITC-anti-rabbit (Vector Laboratories), TRITC-anti-chicken, HRP-anti-mouse, and HRP-anti-rabbit (Jackson ImmunoResearch, West Grove, Pa.).

### Solubility Assays

For solubility assays, skin samples were frozen and ground up, resuspended in a 0.1% Triton X-100-based solution, and nutated for 1 hour at 4° C. (Ihrie et al., supra). The insoluble pellet was lysed in 9M urea buffer. Samples were then analyzed using conventional western blotting protocols.

### Human Tissue Microarrays

Archival paraffin embedded tissue blocks were retrieved from the Dermatopathology Section of the Department of Pathology and Dermatology, University of California, San Francisco and from outside pathology laboratories. Tumor-bearing regions from paraffin-embedded, formalin-fixed tissue samples were identified by a dermatopathologist using routine hematoxylin and eosin stained sections. Tissue microarrays comprising 0.6 mm cores of skin actinic keratoses, skin carcinomas in situ, skin SCCs, and adjacent normal tissue samples were constructed. Sections (5 mm) were cut and placed onto Superfrost plus slides (Fisher Scientific) according to the method of Harradine et al. (2009) Clin. Cancer Res. 15: 5101-5107). 23 AKs, 20 SCClS, and 160 SCCs were analyzed.

### Microarray Analysis

RNA was isolated from the dorsal skin of control and K14CreER;Perp^{fl/fl} mice two weeks post-tamoxifen injection using Trizol (Invitrogen), according to the manufacturer's protocol. RNA samples were processed at the Stanford University Pan Facility and analyzed using Affymetrix Mouse Genome 430 2.0 expression arrays. Probe-level data were processed using BRBArrayTools (Biometric Research Branch of the National Cancer Institute) based on RMA (Robust Multichip Average) for background adjustment, normalization and expression summarization. Class comparison analysis was conducted using SAM (Significance Analysis of Microarrays) with an FDR (False Discovery Rate) of 10% (Tusher et al. (2001) Proc. Natl. Acad. Sci. U.S.A. 98:5116-5121). Gene function categorization based on G0 (Gene Ontology) was carried out using the PANTHER (Protein ANalysis THrough Evolutionary Relationships) classification system.

### Quantitative Reverse Transcription PCR

Skin from control and K14CreER;Perp^{fl/fl} mice was isolated two weeks after tamoxifen injections. RNA was isolated using Trizol (Invitrogen), and 1 mg RNA was reverse transcribed using Moloney Murine Leukemia Virus reverse transcriptase (Invitrogen) and random primers. PCR was performed in triplicate using SYBR green (SA-Biosciences, Frederick, Mass.) and a 7900HT Fast Real-Time PCR machine (Applied Biosystems, Foster City, Calif.), and results were computed relative to a standard curve made with cDNA pooled from all samples. Values were first normalized to bactin and then represented relative to untreated wild-type samples. Five mice per genotype were analyzed. The following primers were used:

β-actin
(SEQ ID NO: 1)
forward 5′-TCC TAG CAC CAT GAA GAT CAA GATC-3′
(SEQ ID NO: 2)
reverse 5′-CTG CTT GCT GAT CCA CAT CTG-3′
S100a9
(SEQ ID NO: 3)
forward 5′-GGA AGG AAG GAC ACC CTG AC-3′
(SEQ ID NO: 4)
reverse 5′-CCA GGT CCT CCA TGA TGT C-3′
Il1f6
(SEQ ID NO: 5)
forward 5′-CTG TTC TGC ACA AAG GAT GG-3′
(SEQ ID NO: 6)
reverse 5′-GCT GCA GAC TCA AAT GTA GAG G-3′
Il22ra
(SEQ ID NO: 7)
forward 5′-GGA CAC CCC GCT TCA CTC-3′
(SEQ ID NO: 8)
reverse 5′-ATT TGG CAA CTC TGG AGG AC-3′
Chi3l1
(SEQ ID NO: 9)
forward 5′-AGCAGT ATT TCT CCA CCC TGA T-3′
(SEQ ID NO: 10)
reverse 5′-CGC TGA GCA GGA GTT TCT CT-3′
Cc120
(SEQ ID NO: 11)
forward 5′-AAC TGG GTG AAA AGG GCT GT-3′
(SEQ ID NO: 12)
reverse 5′-GTC CAA TTC CAT CCC AAA AA-3′

### Results

### Loss of PERP Promotes Tumorigenesis

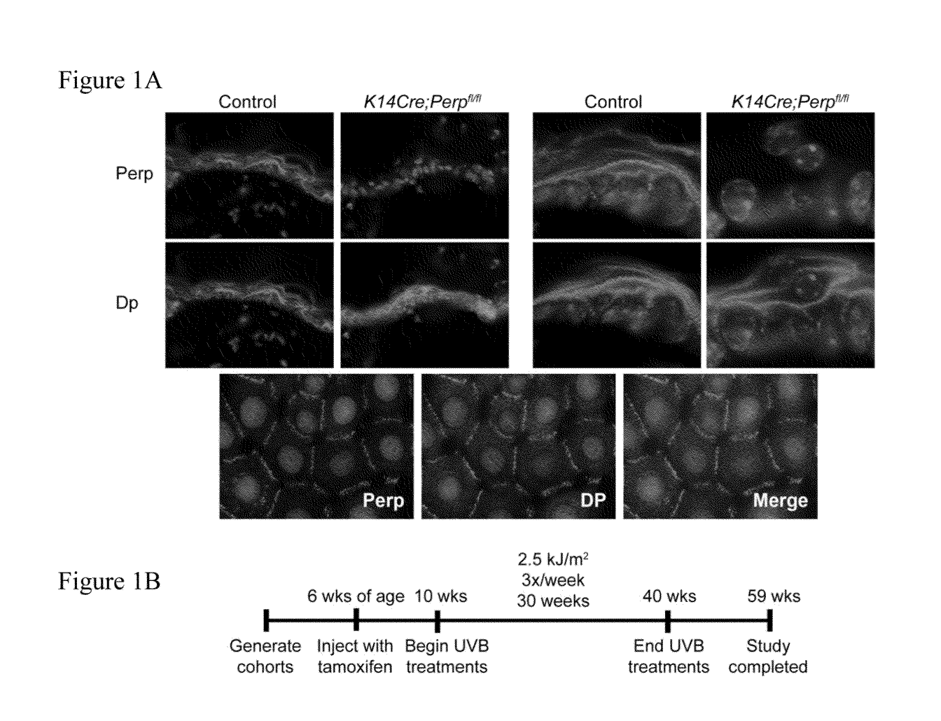

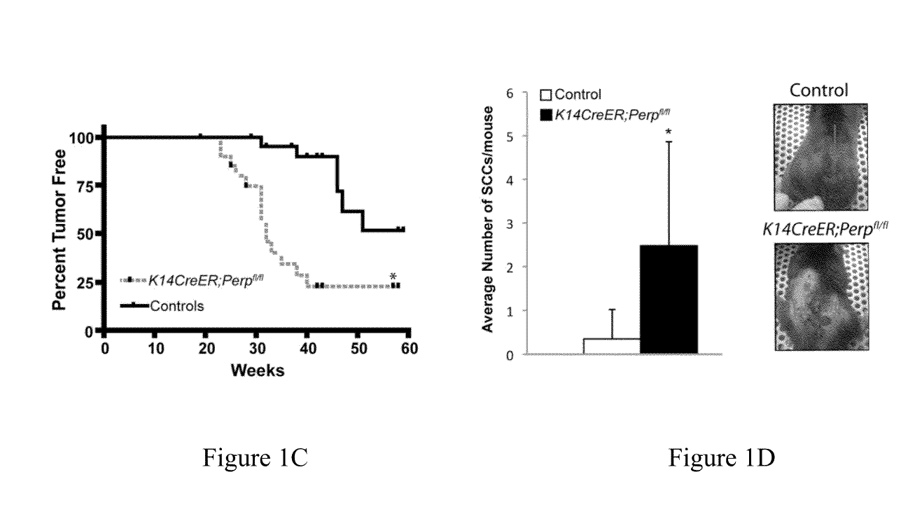

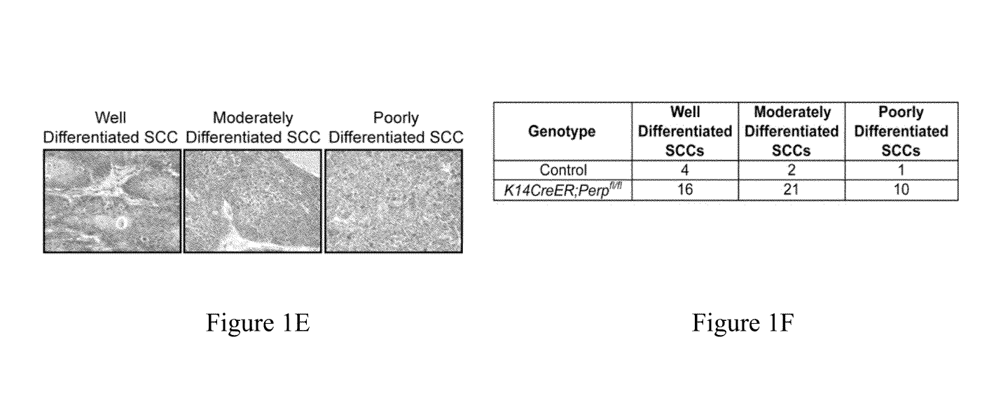

To characterize the function of Perp during tumorigenesis, we examined its role in cancer of the epidermis where it is critical for tissue integrity and homeostasis through its role in desmosomal cell-cell adhesion (Ihrie et al., supra). We examined the tumor predisposition of Perp-deficient mice using a well-defined model for squamous cell carcinoma (SCC) development in which mice are exposed to chronic UVB irradiation (Jiang et al. (1999) Oncogene 18: 4247-4253). This model provides an accurate mimic of human SCC development, which is similarly driven by UVB irradiation. As Perp constitutive null mice die postnatally, we utilized conditional Perp knockout mice (Perp^{fl/fl}; fl=floxed) expressing a tamoxifen-inducible K14CreER transgene to drive tissue-specific deletion of the Perp locus in the epidermis (Indra et al. (2000) Horm. Res. 54: 296-300; Metzger et al. (2005) Methods Mol. Biol. 289: 329-340). Immunofluorescence confirmed that Perp expression was successfully ablated in the epidermis of the majority of these mice 4 weeks after tamoxifen injection (FIG. 1A). To induce SCC development, tamoxifen-treated 10-week old control and Perp^{fl/fl} mice expressing a K14CreER transgene were exposed to chronic treatments (2.5 kJ/m²) of UVB irradiation three times weekly for 30 weeks (FIG. 1B). Kaplan-Meier analysis revealed that mice lacking Perp in the epidermis developed SCCs with reduced average latency (32 weeks) compared to control mice (51 weeks; FIG. 1C). In addition, the average number of SCCs per K14CreER;Perp^{fl/fl} mouse was far greater than in control animals (FIG. 1D). The prominent early tumor development and increased tumor number in Perp-deficient mice compared to controls suggest that Perp loss promotes tumor initiation.

Histological analyses to grade the SCCs according to cellular morphology, invasiveness into the dermis, and overall architecture revealed that SCCs arising in K14CreER;Perp^{fl/fl} mice had a greater propensity to progress to a poorly differentiated stage than tumors arising in control mice, suggesting that Perp loss may also contribute to tumor progression (FIG. 1E, 1F). Despite the presence of invasive tumors, however, no metastases were apparent in the liver or lungs of mice from either cohort (data not shown). Together, these findings indicate that Perp is a key suppressor of skin carcinogenesis and provide the first in vivo demonstration that genetic loss of a desmosomal component can lead to accelerated carcinoma development, facilitating both tumor initiation and progression.

### Perp is an Important Mediator of UVB-Induced Apoptosis

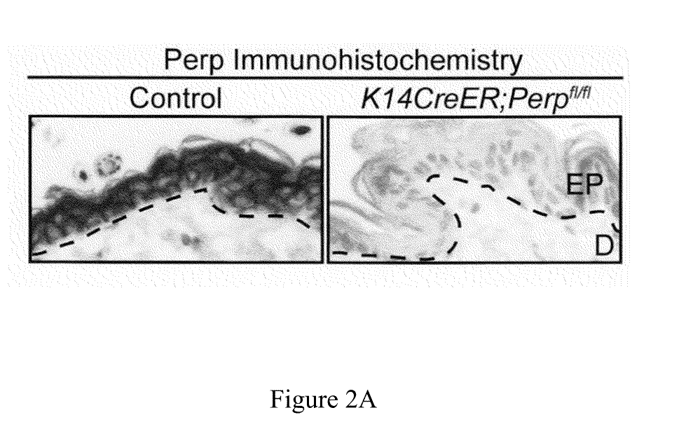

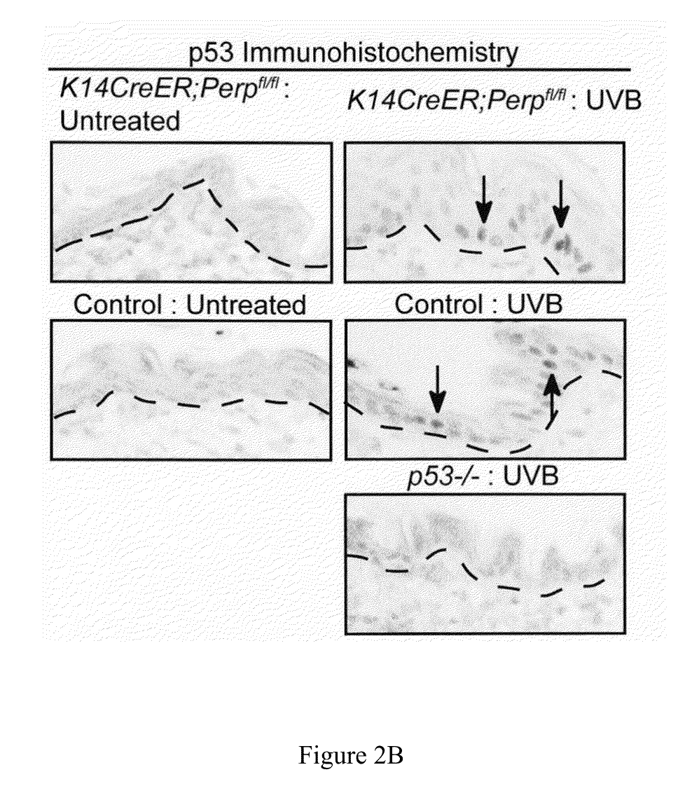

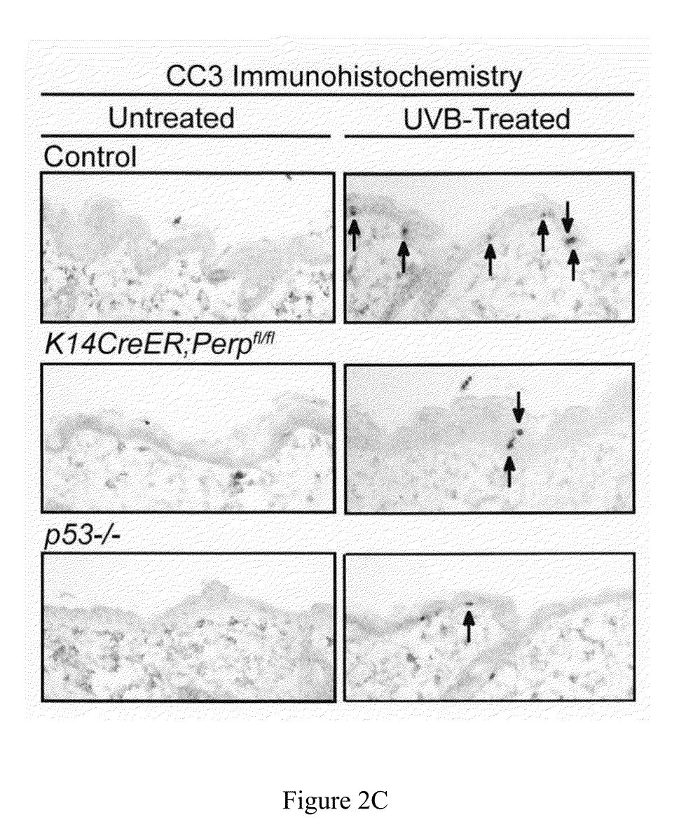

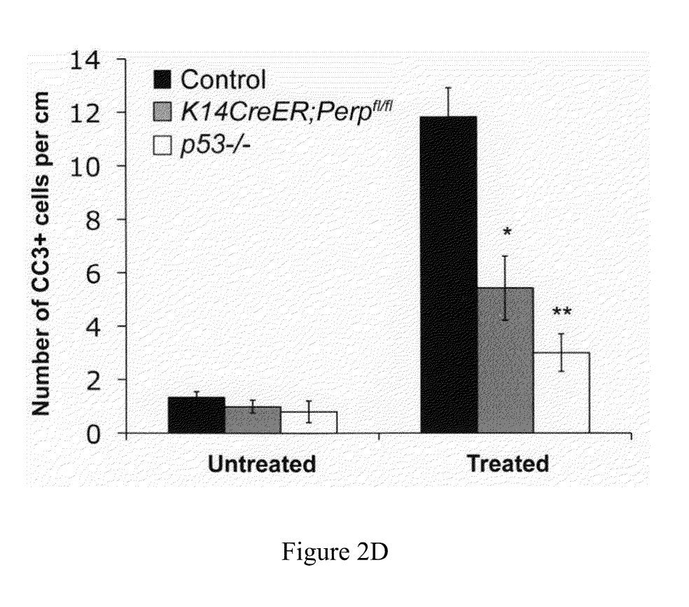

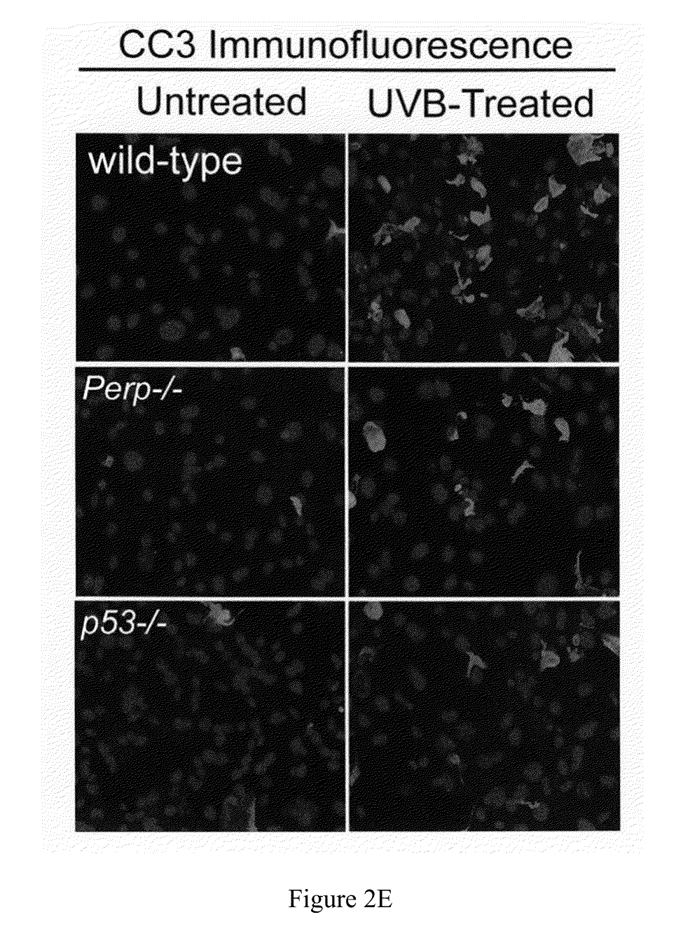

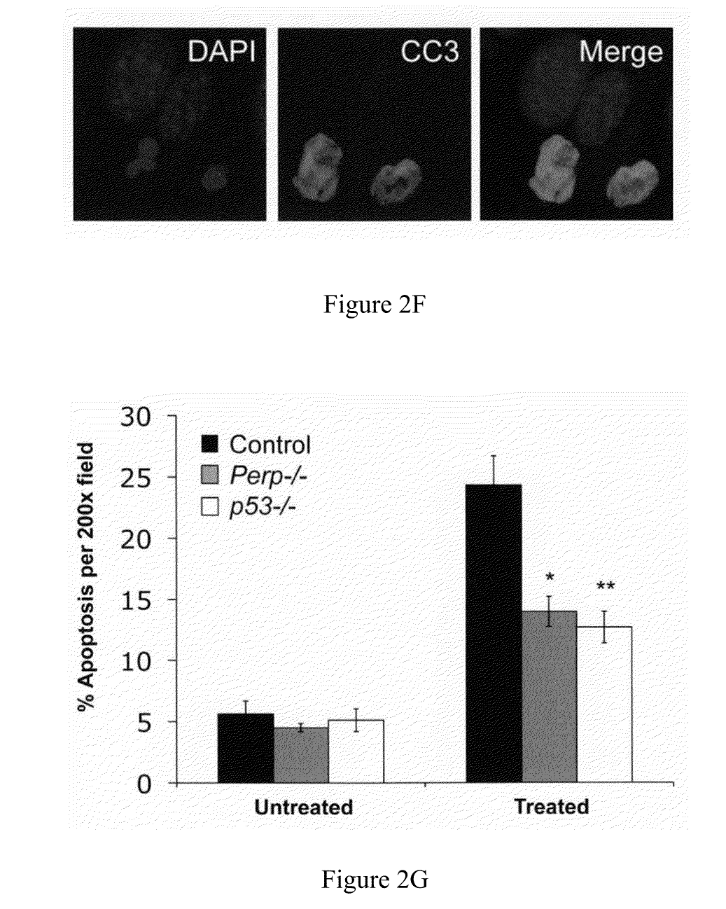

To understand the basis for the tumor development driven by Perp-deficiency, we sought first to determine whether Perp is an important mediator of p53-induced apoptosis in the skin in response to ultraviolet light. Perp plays a cell-type-specific role in p53-mediated apoptosis, being dispensable for apoptosis in fibroblasts but essential for apoptosis of thymocytes and embryonic neurons in response to DNA damage signals (Ihrie et al. (2003) Curr. Biol. 13:1985-1990). To examine Perp's role in p53-dependent apoptosis in keratinocytes, 6-week old K14CreER;Perp^{fl/fl} and control mice were injected with tamoxifen (which ablated Perp expression in K14CreER;Perp^{fl/fl} mice; FIG. 2A), and 4 weeks later, mice were exposed to one dose of 2.5 kJ/m² of UVB radiation. We confirmed that p53 is induced 24 hours after UVB treatment in the epidermis of both wild-type and K14CreER;Perp^{fl/fl} mice (FIG. 2B). Apoptotic indices were then determined by quantifying the number of cleaved Caspase 3-positive cells in the epidermis. Minimal apoptosis was detected in untreated skin of mice of all genotypes (FIG. 2C, 2D). While robust apoptosis was observed in the epidermis of wild-type mice in response to UVB radiation, p53 null mice displayed significantly attenuated levels of apoptosis (FIG. 2C, 2D). Analysis of the epidermis of Perp deficient mice also revealed diminished apoptosis levels, to an extent nearly equivalent to p53 loss (FIG. 2C, 2D). We confirmed these findings by assaying apoptosis in differentiated keratinocytes in vitro, through analysis of both cleaved Caspase 3 positivity and classical apoptotic nuclear morphology and condensed chromatin by DAPI staining (Hakem et al. (1998) Cell 94:339-352; Joza et al. (2001) Nature 410: 549-554). We found that Perp−/− keratinocytes displayed defective apoptosis in response to UVB, similar to p53−/− keratinocytes (FIG. 2E-2G). Together, these results demonstrate that Perp plays an important role in UVB-induced apoptosis in both the skin in vivo and keratinocytes in vitro. As p53 inactivation is proposed to promote UVB-induced carcinogenesis by allowing inappropriate survival of cells sustaining UVB-induced damage (Ziegler et al. (1994) Nature 372: 773-776), enhanced survival of Perp-deficient cells after exposure to ultraviolet light could similarly enable tumor initiation.

### Desmosome Components are Selectively Downregulated During Tumorigenesis

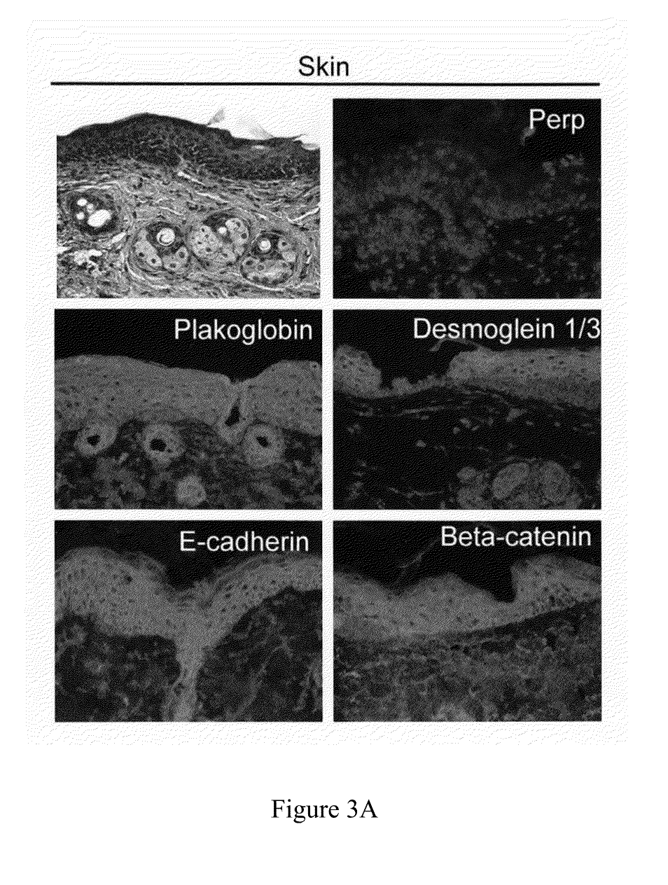

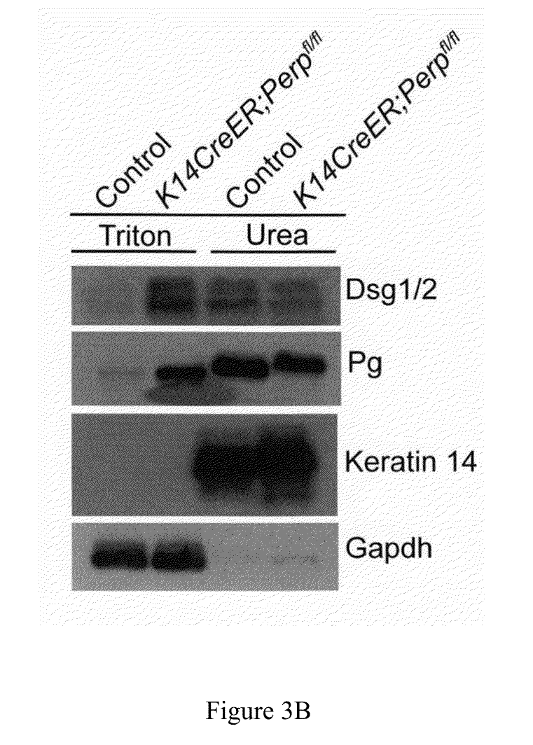

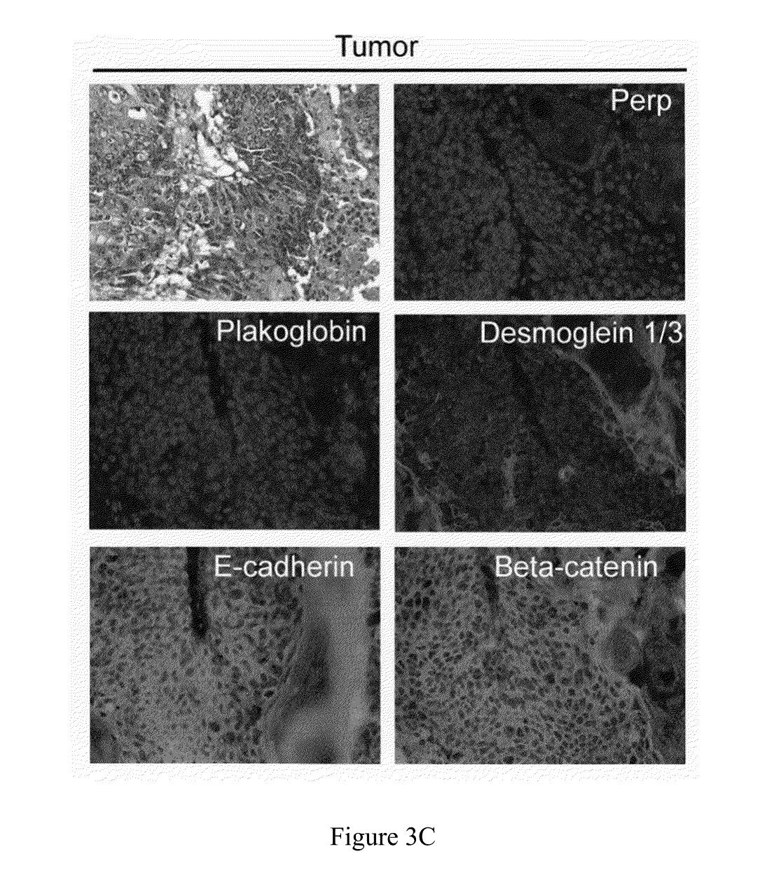

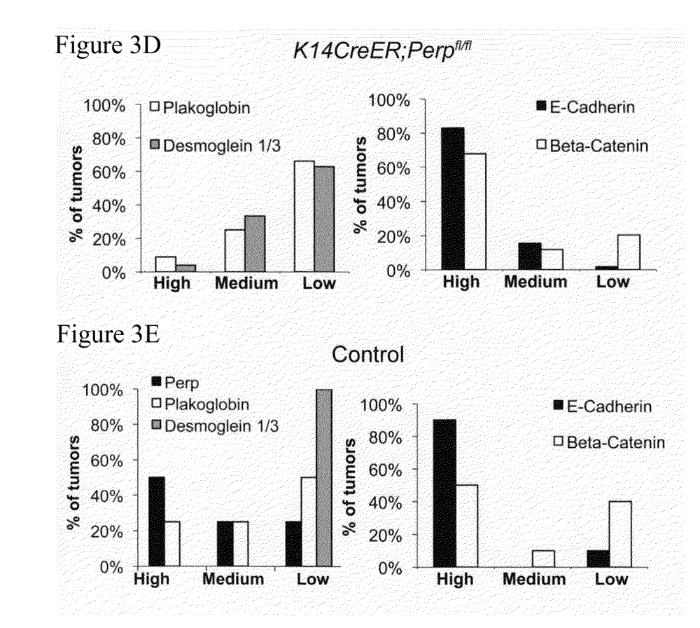

As a key desmosomal constituent in the epidermis, Perp loss could also promote cancer through effects on cell-cell adhesion. In constitutive Perp knockout mice, Perp loss does not abrogate desmosome formation, but leads to decreased numbers of desmosomes and reduced stability of desmosomal components (Ihrie et al., supra). Simple immunofluorescence analysis of desmosome proteins in Perp−/− newborn skin or K14CreER;Perp^{fl/fl} adult mouse skin does not show perturbed membrane localization of desmosomal components (Ihrie et al., supra), FIG. 1A, FIG. 3A), and thus it is necessary to use a biochemical assay to show that desmosomes are functionally compromised upon Perp loss. This solubility assay is based on the fact that properly formed desmosomal complexes can only be solubilized by chaotropic agents, whereas improperly assembled desmosomal components can be solubilized by the nonionic detergent Triton X-100 (Bornslaeger et al. (2001) J. Cell Sci. 114: 727-738). We found that the desmosomal constituents Desmoglein 1/2 and Plakoglobin displayed enhanced Triton X-100-solubility in skin from K14CreER;Perp^{fl/fl} mice compared to skin from control mice (FIG. 3B), confirming that acute deletion of Perp leads to impaired desmosome function similar to that observed in constitutive Perp−/− mice (Ihrie et al., supra).

To determine whether Perp ablation might facilitate tumorigenesis by promoting complete desmosome loss, tumors from Perp deficient mice were analyzed for the expression of Desmoglein 1/3 and Plakoglobin. Both the percentage of epithelial cells expressing each marker at the plasma membrane and the intensity of staining were measured. Staining for each antigen in tumors was categorized as high (>70%), medium (30-70%), or low (<30%) level expression. Analysis of the tumors in the K14CreER;Perp^{fl/fl} mice revealed that the majority of lesions expressed low levels of desmosome components, suggesting that desmosome dissolution had occurred (FIG. 3C, 3D). Thus, complete desmosome destabilization can occur during tumorigenesis.

To assess whether this loss of desmosomal component expression reflected a general change in differentiation status of the cells during tumorigenesis, such as Epithelial to Mesenchymal Transition (EMT) (Kalluri et al. (2009) J. Clin. Invest. 119: 1420-1428), we stained tumors for markers of adherens junctions. Most cells in the tumors displayed robust membrane staining for both E-cadherin and Beta-catenin, suggesting that adherens junctions remained intact (FIG. 3C, 3D). The observation that adherens junctions are maintained suggests that desmosome loss does not promote tumorigenesis through a general trans-differentiation mechanism, but rather through a more specific mechanism related to changes caused by complete desmosome-deficiency. Accordingly, staining of tumors arising in the Perp-deficient mice for Smooth Muscle Actin and Keratin 8 revealed a lack of expression of these markers (data not shown). Together, these data indicate that Perp loss can facilitate desmosome downregulation and that direct loss of desmosomes contributes to tumor development, but in a manner distinct from that of adherens junction dysfunction.

We also sought to establish whether desmosome destabilization occurred during SCC development in control animals by staining tumors from these mice for desmosomal proteins. Whereas nonlesional skin in the control mice exhibited normal expression of Perp, Desmoglein 1/3, and Plakoglobin (data not shown), all of the tumors from these mice displayed low level expression of one or more desmosomal components (FIG. 3E). Desmoglein 1/3 appeared to be lost most commonly. Since desmosomes were intact at the beginning of the study, these data further suggest that targeted downregulation of the desmosome is an active characteristic of tumor development, and that weakened desmosomal adhesion, as in the case of Perp-deficiency, facilitates this downmodulation. In addition, analysis of adherens junction components in control tumors revealed intact E-cadherin and Beta-catenin expression at the plasma membrane, similar to tumors in the K14CreER;Perp^{fl/fl} mice (FIG. 3E). Thus, downregulation of desmosomal constituents with maintenance of adherens junctions is a general feature of SCC development.

### Perp Expression is Downregulated in Human SCCs

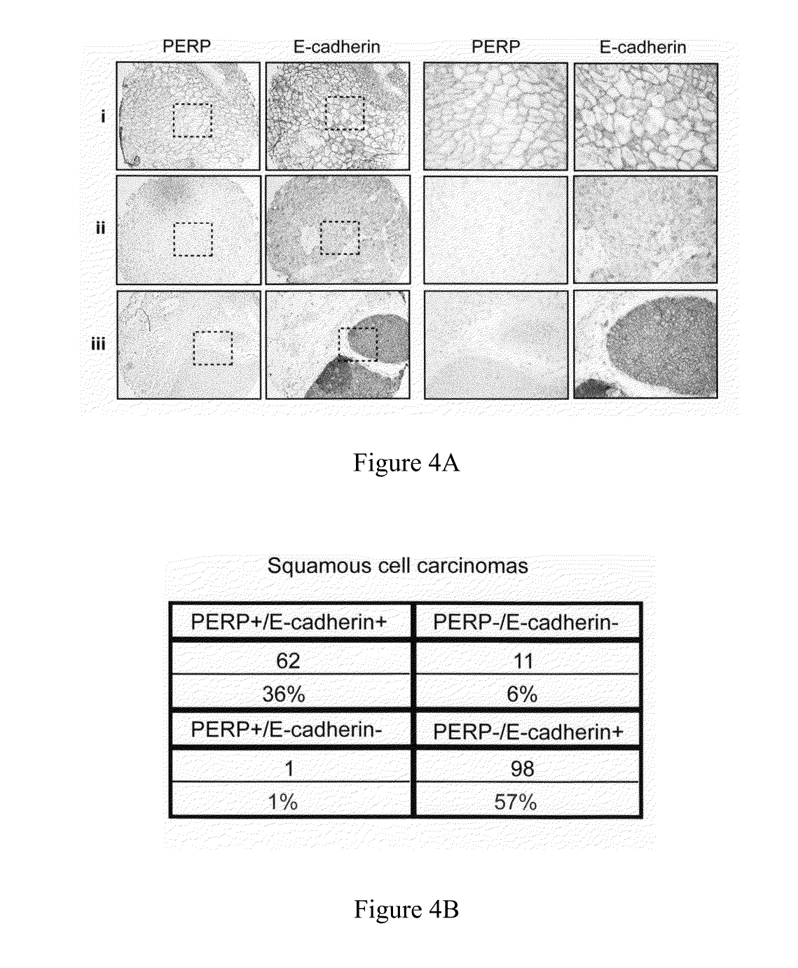

Our findings suggest that desmosome loss, coupled with maintenance of adherens junctions, may represent an important stage of tumorigenesis. To determine the relevance of this observation to human SCC development, we stained a panel of samples reflecting different stages of human skin SCC development, ranging from actinic keratoses to moderately differentiated SCCs, for both PERP and E-cadherin. Each sample was scored based on the percentage of epithelial cells exhibiting membrane staining in addition to the intensity of the membrane staining in the epithelial portion of each tumor. Samples with greater than 10% of the epithelial cells expressing intense membrane staining were given a positive score, while those with less than 10% of the epithelial cells expressing either PERP or E-cadherin were given a negative score. We first noted a significant decline in the percentage of samples displaying Perp expression in the transition between AKs and SCCIS, suggesting that Perp expression is downregulated during tumor progression (p=0.049, z-test). Interestingly, while a subset of all tumors displayed intact PERP and E-cadherin expression and another group showed complete lack of expression of both PERP and E-cadherin, the majority of tumors (57%) lacked PERP expression while retaining E-cadherin expression, akin to our observations in the mouse SCC model (FIG. 4A, 4B). These findings suggest that PERP loss with retention of E-cadherin represents a significant phase of human tumorigenesis. Moreover, since the vast majority of all SCCs examined (93%) retain E-cadherin expression, and since E-cadherin loss is thought to be a late event in tumorigenesis, our data suggest a temporal sequence whereby PERP loss occurs before E-cadherin loss in the progression of human SCC.

### Perp Ablation Induces an Inflammatory Gene Signature

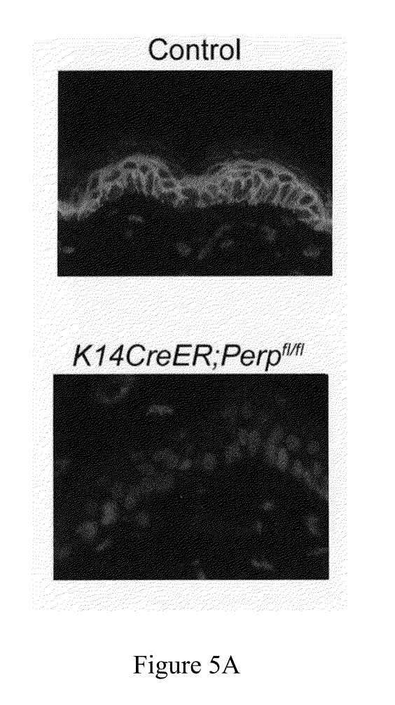

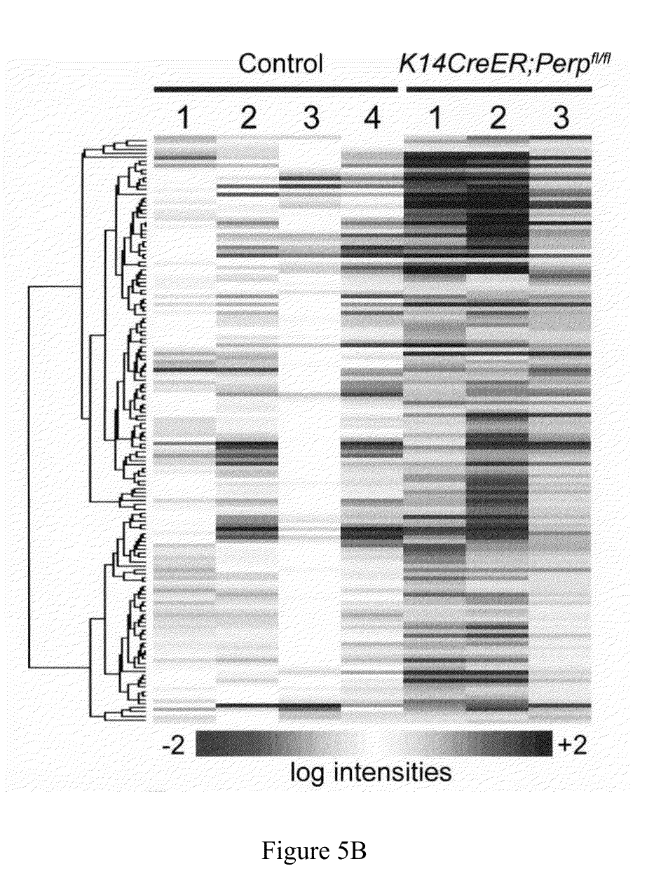

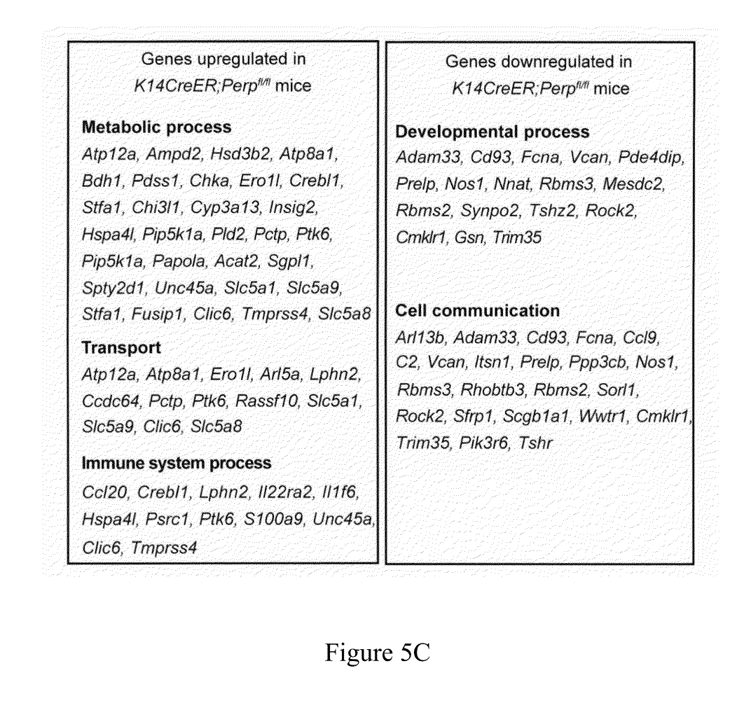

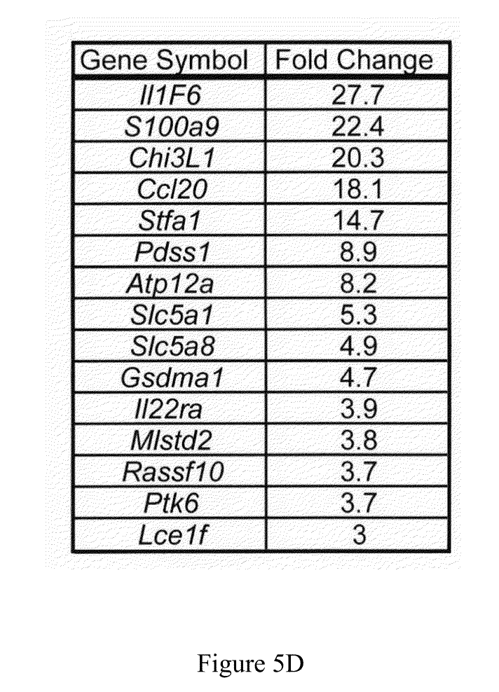

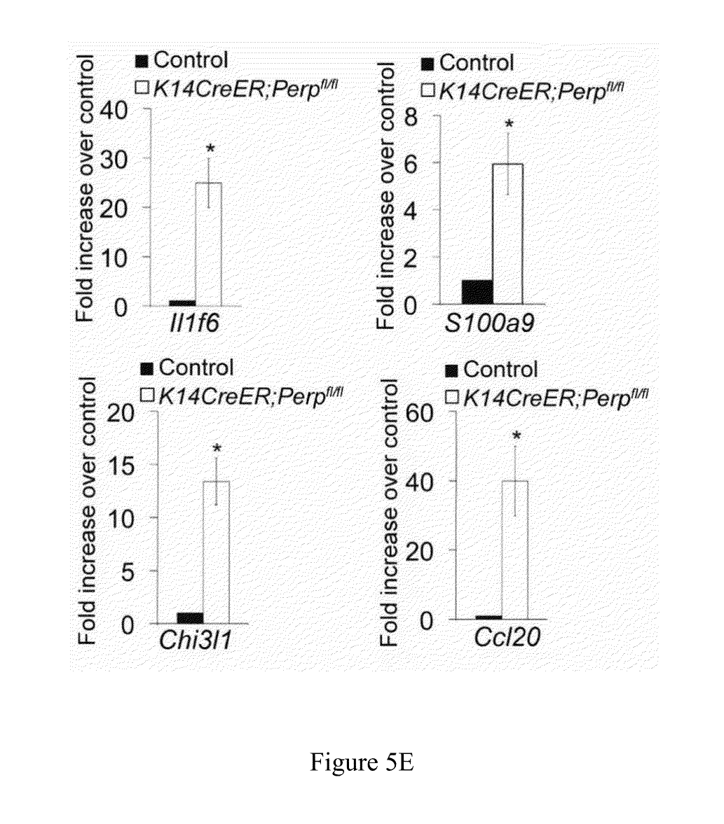

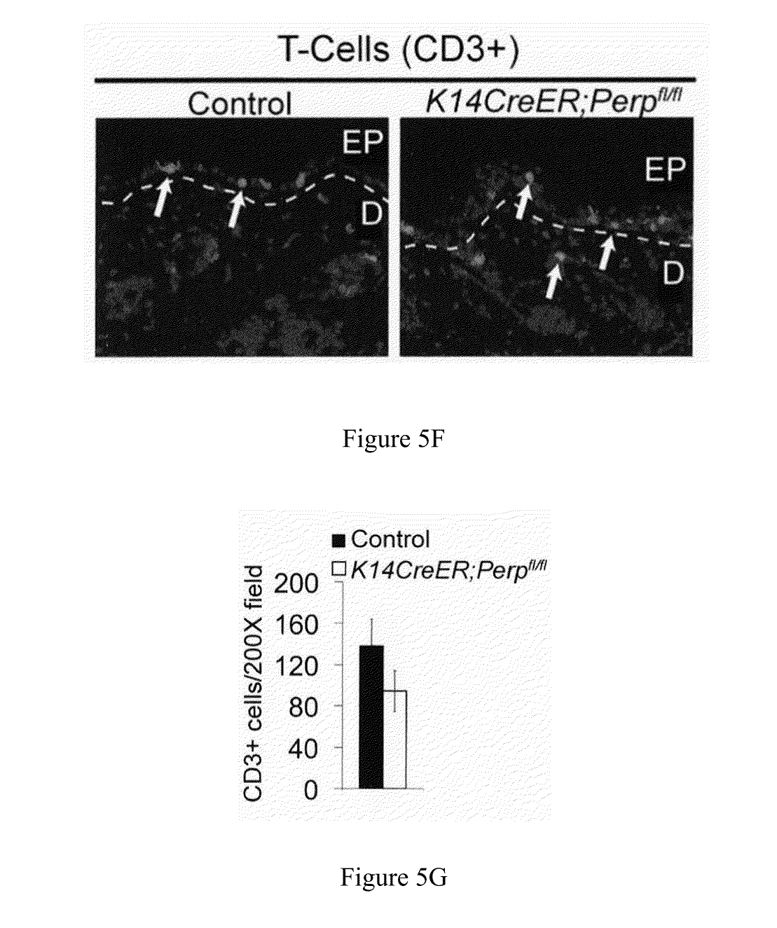

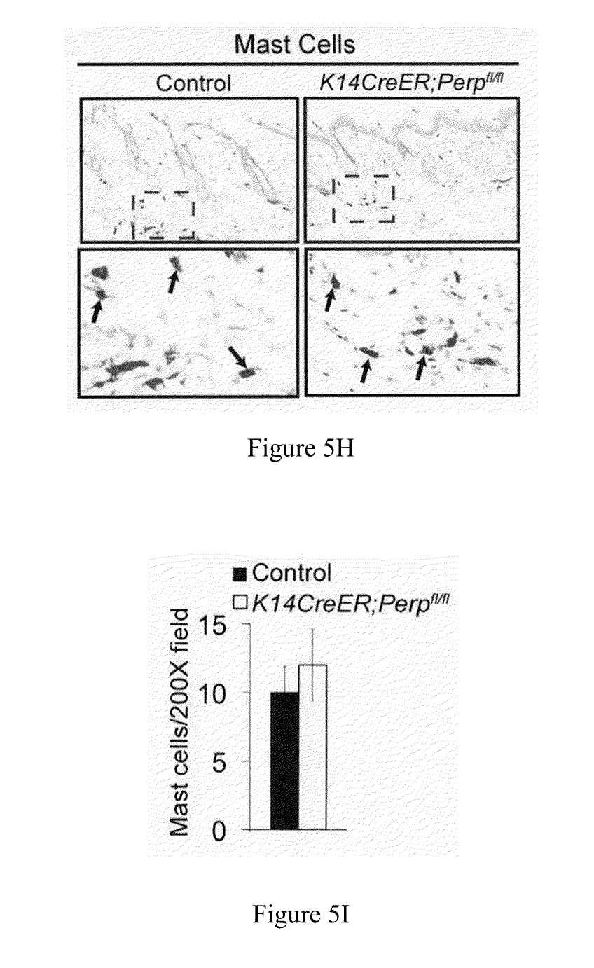

To understand how Perp-deficiency might cooperate with chronic UVB exposure to promote cancer, we performed microarray analyses to identify those genes whose expression is altered upon Perp loss. Cohorts of K14CreER;Perp^{fl/fl} and control mice were generated and skin was processed for RNA analysis two weeks post tamoxifen injection, a timepoint at which we could first detect Perp protein expression loss throughout the epidermis (FIG. 5A). Using Significance Analysis of Microarrays (SAM) according to the method of Tusher et al. (Proc. Natl. Acad. Sci. U.S.A. (2001) 98:5116-5121), we identified a panel of 143 genes that were differentially regulated in the K14CreER;Perp^{fl/fl} mice compared to controls (FDR=10%; FIG. 5B). These included 51 upregulated and 92 downregulated genes.

We next classified the genes changing with altered Perp status based on Gene Ontology functional annotation. The three largest statistically significantly enriched groups of genes upregulated upon Perp ablation were in the categories of metabolic process, transport, and immune system process (FIG. 5C). Significantly enriched classes of genes downregulated upon Perp loss included those linked to developmental processes and cell communication (FIG. 5C). To identify genes whose induction might promote cancer, we examined those genes that were most highly upregulated in the absence of Perp. Upon examination of the list of genes induced 3-fold or greater in the absence of Perp, we discovered that the most highly induced were several inflammation-related genes (FIG. 5D). These included: Interleukin 1 Family member 6 (Il1f6), an inflammatory cytokine sufficient to induce an inflammatory response when expressed in keratinocytes of transgenic mice (Blumberg et al. (2007) J. Exp. Med. 204: 2603-2614); S100a9, a calcium binding protein with cytokine-like function in inflammation and cancer (Gebhardt et al. (2006) Biochem. Pharmacol. 72: 1622-1631; Salama et al. (2008) Eur. J. Surg. Oncol. 34: 357-364); Chitinase 3-like 1 (Chi311), a mammalian chitinase involved in enhancing inflammation as well as angiogenesis and extracellular matrix remodeling, thereby promoting tumorigenesis (Eurich et al. (2009) World J. Gastroenterol. 15: 5249-5259); and Chemokine ligand 20 (Ccl20), an established chemoattractant for subsets of lymphocytes and dendritic cells which is also known to promote tumor growth (Beider et al. (2009) PLoS One 4:e5125. doi:10.1371/journal.pone.0005125; Hasan et al. (2006) J. Immunol. 176: 6512-6522; Punj et al. (2009) Blood 113:5660-5668; and Ben-Baruch (2006) Cancer Metastasis Rev. 25: 357-371). Additionally, this list included 1122ra, a class II cytokine receptor and mediator of innate immune responses (O'Shea et al. (2008) Immunity 28: 477-487). Quantitative RT-PCR analysis verified that the expression of these genes is indeed significantly induced in the skin of Perp-deficient mice (FIG. 5E, data not shown). Chronic UVB exposure combined with Perp loss leads to the recruitment of immune cells to the skin.

The induced inflammatory gene signature in Perp-deficient mice could reflect gene expression changes intrinsic to keratinocytes, or, alternatively, the recruitment of inflammatory cells to the skin of Perp-deficient mice. To distinguish these possibilities, we analyzed untreated K14CreER;Perp^{fl/fl} and control skin samples for the presence of inflammatory cells. Histological staining for T cells, mast cells, and myeloid cells indicated no difference in immune cell numbers between the Perp-ablated samples and the controls (FIG. 5F-5I, data not shown), suggesting that Perp deficiency induces an inflammatory gene expression program directly in keratinocytes rather than causing recruitment of immune cells to the skin. The observation that Perp loss triggers the induction of genes known to promote inflammation, combined with the fact that inflammation is causally linked to cancer development (Grivennikov et al. (2010) Cell 140: 883-899), provides a rationale for how Perp-deficiency could contribute to cancer.

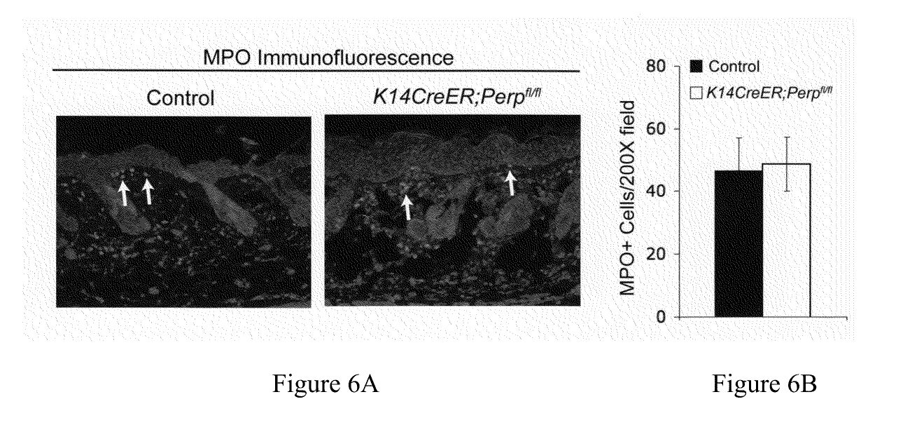

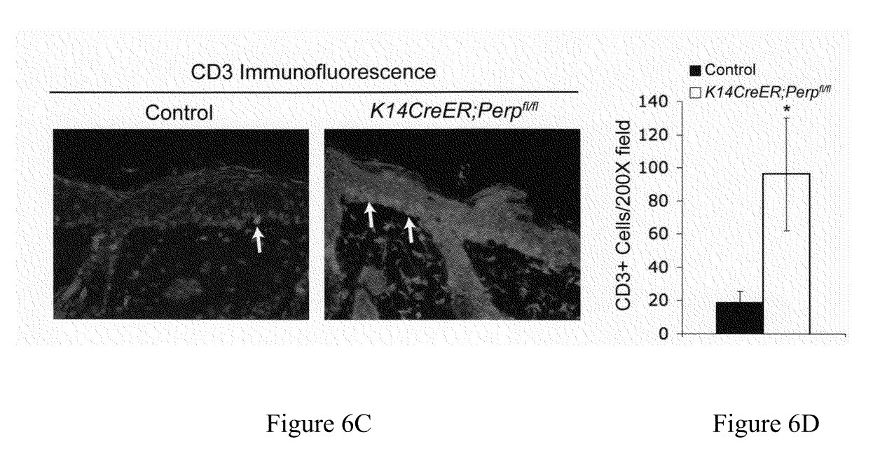

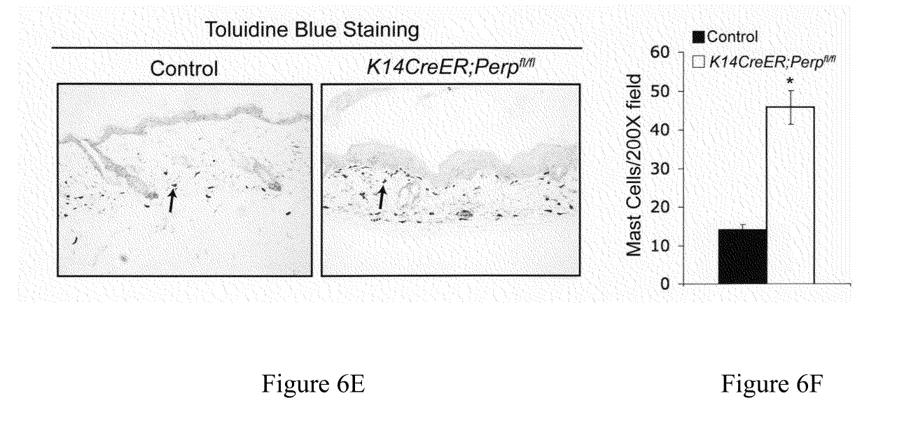

We hypothesized that persistent cytokine/chemokine signaling in K14CreER;Perp^{fl/fl} mice, combined with chronic UVB exposure, might ultimately attract immune cells, thereby promoting tumor formation. To investigate this idea, we queried the presence of inflammatory cells in the skin from a subset of control and K14CreER;Perp^{fl/fl} mice at an intermediate timepoint in the tumor study, after 19 weeks of chronic UVB treatment, by quantifying numbers of myeloid cells, T-cells, and mast cells (FIG. 6A-6F). While we did not detect any differences in the number of myeloid cells, assessed by MPO-positivity (FIG. 6A, 6B; Pinkus et al. (1991) Mod. Pathol. 4: 733-741), we did observe increased numbers of T-cells present throughout the skin of K14CreER;Perp^{fl/fl} mice compared to controls (FIG. 6C, 6D). Moreover, we noted a striking increase in mast cell numbers in the skin from K14CreER;Perp^{fl/fl} mice relative to controls (FIG. 6E, 6F). As mast cells have been reported to surround tumors in a variety of cancers, including SCCs (Ribatti et al. (2001) Br. J. Haematol. 115: 514-521; Coussens et al. (1999) Genes Dev. 13: 1382-1397; Ch'ng et al. (2006) Mod. Pathol. 19: 149-159; Meininger (1995) Chem. Immunol. 62: 239-257), and because they have been shown to play a key role in promoting tumorigenesis through the secretion of factors that remodel the tumor microenvironment and stimulate angiogenesis (Meininger, supra; Maltby et al. (2009) Biochim. Biophys. Acta 1796: 19-26; Bashkin et al. (1990) Blood 75: 2204-2212; Vlodaysky et al. (1992) Invasion Metastasis 12: 112-127), their presence in the UVB-treated Perp-deficient mouse skin suggests an additional mechanism through which Perp loss may stimulate tumorigenesis.

### Discussion

The importance of disrupted cell-cell adhesion for cancer development is underscored by the observed downregulation of adherens junction components during human cancer progression and genetic experiments demonstrating tumor prone phenotypes of mice deficient for E-cadherin, Alpha-catenin, or p120-catenin, components of the adherens junction [Davis et al. (2006) Dev. Cell 10: 21-31; Perez-Moreno et al. (2008) Proc. Natl. Acad. Sci. U.S.A. 105: 15399-15404; Vasioukhin et al. (2001) Cell 104: 605-617). In contrast, the data regarding desmosome protein expression during human cancer progression are conflicting (Yashiro et al. (2006) Eur. J. Cancer 42: 2397-2403; Roepman et al. (2005) Nat. Genet. 37: 182-186; Papagerakis et al. (2009) Hum. Pathol. 40:1320-1329; Depondt et al. (1999) Eur. J. Oral Sci. 107: 183-193; Furukawa et al. (2005) Cancer Res. 65: 7102-7110; Chen et al. (2007) Oncogene 26: 467-476; Kurzen et al. (2003) J. Cutan. Pathol. 30: 621-630), and the contribution of desmosome dysfunction to cancer development has not been clearly established using in vivo mouse models.

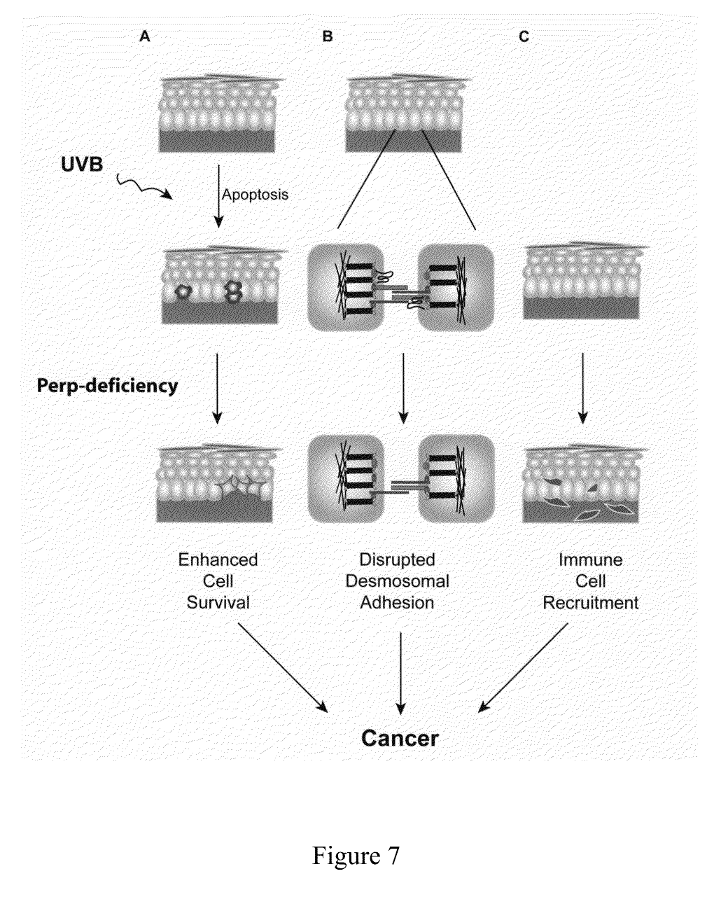

Here, we show that loss of the desmosomal component Perp predisposes mice to UVB induced SCC development by enhancing both tumor initiation and progression. The effects of Perp ablation are at multiple levels, leading both to compromised apoptosis in response to ultraviolet light and loss of desmosomal adhesion (FIG. 7). The defective apoptosis could allow the inappropriate survival of damaged cells, which could help initiate tumors. The exact mechanism through which Perp promotes apoptosis remains to be elucidated, but it may relate to Perp function at the desmosome, as apoptotic defects were previously reported in cells lacking either the desmosomal component desmoglein 1 or desmoglein 2 [Nava et al. (2007) Mol. Biol. Cell 18: 4565-4578; Dusek et al. (2006) J. Biol. Chem. 281: 3614-3624). In addition, compromised UVB-induced apoptosis in the epidermis has also been observed in mice lacking the p53 target gene Noxa, a member of the Bcl-2 family (Naik et al. (2007) J. Cell Biol. 176: 415-424). Our findings indicate that Noxa is insufficient to drive apoptosis in the absence of Perp, and therefore that Perp and Noxa may collaborate to cause apoptosis. In addition, we observe perturbations in desmosomal adhesion. Interestingly, the desmosome downregulation we observe in tumors occurs without adherens junction loss or other signs of EMT, highlighting a specific role for desmosome loss in tumor development. It may be that desmosome loss occurs during early stages of tumorigenesis, facilitating early cancer progression, and that adherens junctions are lost subsequently, thereby promoting invasion and metastasis phenotypes. Our analysis of human SCC samples supports the idea that PERP-deficient, E-cadherin positive samples reflect an important stage of human skin carcinogenesis.

To understand further how Perp-deficiency might enhance tumor development, we examined gene expression profiles upon Perp loss. Several of the genes induced upon Perp inactivation are known to be involved in promoting inflammation and tumorigenesis (Blumberg et al. (2007) J. Exp. Med. 204: 2603-2614; Gebhardt et al. (2006) Biochem. Pharmacol. 72: 1622-1631; Salama et al. (2008) Eur. J. Surg. Oncol. 34: 357-364; Eurich et al. (2009) World J Gastroenterol 15: 5249-5259; Beider et al. (2009) PLoS One 4: e5125. doi:10.1371/journal.pone.0005125; Hasan et al. (2006) J. Immunol. 176: 6512-6522; Punj et al. (2009) Blood 113:5660-5668; Ben-Baruch (2006) Cancer Metastasis Rev. 25: 357-37; O'Shea et al. (2008) Immunity 28: 477-487). Inflammation is a well-established causative factor in tumorigenesis (Grivennikov et al. (2010) Cell 140: 883-899), as evidenced by tumor prone mouse strains deficient for specific subsets of immune or inflammatory cells exhibiting reduced tumor burdens (Coussens et al. (1999) Genes Dev 13: 1382-1397; Andreu et al. (2010) Cancer Cell 17: 121-134). The induction of a set of inflammation-associated genes presents a plausible explanation for how Perp-deficiency can promote cancer in cooperation with chronic UVB damage. Indeed, we found that Perp-deficiency, in conjunction with chronic UVB exposure, led to the infiltration of T-cells and mast cells. Mast cells can clearly promote tumorigenesis (Maltby et al. (2009) Biochim. Biophys. Acta 1796: 19-26; Bashkin et al. (1990) Blood 75: 2204-2212; Vlodaysky et al. (1992) Invasion Metastasis 12: 112-127), and their accumulation in the UVB-treated, Perp-deficient epidermis provides another mechanism through which Perp loss can stimulate cancer development (FIG. 7).

Our studies also provide insight into mechanisms of p53-mediated tumor suppression in skin cancer. The relevance of p53 in skin cancer development is highlighted by the observations that p53 is mutated in at least 90% of human SCCs (Benjamin et al. (2008) Photochem. Photobiol. 84: 55-62) and that p53 null mice display an enhanced predisposition to UVB-triggered skin cancer (Jiang et al. (1999) Oncogene 18: 4247-4253; Bruins et al. (2004) Mol. Cell Biol. 24: 8884-8894). While p53's ability to drive apoptosis in response to ultraviolet light has been shown to limit SCC formation (Melnikova et al. (2005) Mutat. Res. 571: 91-106), the molecular pathways underlying p53's tumor suppressor properties are incompletely understood. p53 is a transcriptional activator, but the genes mediating p53 tumor suppressor function have been unclear, as none of the mouse strains deficient for p53 target genes exhibits a spontaneous tumor predisposition (Lozano et al. (2005) J. Pathol. 205: 206-220). Instead, analysis of target genes in specific contexts may reveal key functions as mediators of p53 tumor suppressor function. This notion is exemplified by studies of the apoptotic target gene Puma, which is important for p53 tumor suppression in the setting of Em-myc driven B-cell lymphomas (Hemann et al. (2004) Proc. Natl. Acad. Sci. U.S.A. 101: 9333-9338). Our studies have provided important novel insights into pathways of p53 tumor suppression by suggesting that Perp is a critical mediator of p53 tumor suppressor function in UVB-induced SCC development. In addition, although the role of p63 in cancer has been more controversial, Perp loss could also potentially explain how tumors might arise in the absence of p63.

Non-melanoma skin cancer is one of the most common malignancies in the US (Armstrong et al. J. Photochem. Photobiol. B 63: 8-18). Fortunately, identifying SCC lesions before they progress into poorly differentiated tumors is aided both by facile detection and increased awareness of the consequences of chronic sun exposure. However, this is not the case for other stratified epithelia-derived cancers such as head and neck or esophageal cancers, which have poor survival rates (Detailed Guide: Esophagus Cancer (2009) American Cancer Society). Our findings may provide a framework to better understand how these more deadly diseases progress. Indeed, the loss of desmosomal component expression with maintenance of adherens junction expression observed in the K14CreER;Perp^{fl/fl} mouse tumors and in human skin SCCs was recapitulated in samples derived from humans with head and neck SCCs (data not shown).

The idea that desmosome loss may precede adherens junction loss could have important clinical implications. While E-cadherin status can provide a useful prognostic indicator for a variety of epithelial cancers, loss of this marker is associated with late-stage tumor progression (Schipper et al. (1991) Cancer Res. 51: 6328-6337; Kashiwagi et al. (2010) Br. J. Cancer 103: 249-255; Mell et al. (2004) Clin. Can. Res. 10: 5546-5553). Identifying markers like PERP that are altered earlier during tumorigenesis could potentially enhance diagnosis, grading, and prognostication, leading to more informed choices of therapy. The increased frequency of advanced tumors in the Perp-deficient mice relative to controls supports the idea that human tumors lacking PERP may ultimately progress more aggressively, and thus that PERP status may provide a useful diagnostic or prognostic marker.

### Example 2

### Evaluation of PERP as a Diagnostic and Prognostic Marker in a Clinical Study of Oral Cavity Carcinogenesis

### Case Set Selection

We used formalin-fixed, paraffin-embedded tissues from 32 patients with oral cavity (OC) SCC (25 oral tongue, 3 floor of mouth, 2 retromolar trigone, and 1 buccal) and HPV(−) oropharyngeal (OP) SCC (1 base of tongue) to construct the first tissue microarray (TMA1). All patients had clinical follow-up (median 57.2 months, range 4.3-107.4 months) and were identified from the Stanford Department of Radiation Oncology Clinical Database. Table 1 shows the patient distribution and treatment types. Most of the patients had OC primary tumors and only 1 had an OP tumor. All cases were negative for p16 (clone E6H4, predilute, Tris, MTM Laboratories), a surrogate marker for high-risk HPV. TMA1 was constructed with cores of normal mucosa, CIS, invasive tumor and nodal metastasis from the same cases whenever available. For 6 patients, cores of tumor and normal mucosa were also taken from a local recurrence or new primary OC cancer and included in the TMA.

The validation TMA (TMA2) was constructed from formalin-fixed, paraffin embedded specimens from 34 patients ranging in age from 34-85 years (mean 58 years) with oral epithelial dysplasia (17 mild, 9 moderate and 8 severe), who were identified from the University of California San Francisco (UCSF) Oral Pathology archives. Insufficient clinical follow up was available to permit clinical outcomes analyses for this set of patients.

TABLE 1
Patient distribution of 32 patients with invasive SCC (TMA 1)

| Characteristic | Descriptor | N (%) |
|---|---|---|
| Age (median: 57) | <60 | 18 (56.2) |
| | ≧60 | 14 (43.8) |
| Gender | Male | 12 (37.5) |
| | Female | 20 (62.5) |
| Tumor site | Oral cavity | 30 (93.8) |
| | Oropharynx | 2 (6.2) |
| T-Classification | 1-2 | 24 (75) |
| | 3-4 | 8 (25) |
| N-Classification | 0 | 22 (68.8) |
| | 1-2a | 7 (21.9) |
| | 2b-c | 3 (9.3) |
| Treatment | Surgery alone | 15 (46.9) |
| | Surgery & RT +/− chemo | 15* (46.9) |
| | Chemoradiotherapy | 2 (6.2) |

*Postoperative radiotherapy (RT) alone in 12, postoperative chemoradiotherapy in 3

### TMA Construction

Two separate tissue microarrays (TMA) were constructed by using a tissue arrayer (Beecher Instruments, Silver Spring, Md.) to create new paraffin blocks from representative 1.0 mm cores taken in duplicate (Liu et al. (2002) Am. J. Pathol. 161(5):1557-65). TMA1 was created from the Stanford samples and included two or more cores of normal squamous mucosa from 33 samples, carcinoma in situ (CIS) from 21 samples, invasive SCC from 35 samples, and metastatic carcinoma within regional lymph nodes from 14 samples. Table 2 shows the number of evaluable cores. TMA2 contained duplicate cores of the oral epithelial dysplasia samples from UCSF. All cores for TMA2 were evaluable.

TABLE 2
Number (%) of evaluable cores for TMA 1

| | | | Invasive | Nodal |
|---|---|---|---|---|
| | Normal | | Carcinoma | Metastasis |
| | Mucosa (%) | CIS (%) | (%) | (%) |
| PERP Extent | 30/33 (90.9) | 18/21 (85.7) | 33/35 (94.3) | 13/14 (92.9) |
| PERP Intensity | 31/33 (93.9) | 18/21 (85.7) | 34/35 (97.1) | 14/14 (100) |
| E-cadherin | 31/33 (93.9) | 17/21 (81.0) | 31/35 (88.6) | 14/14 (100) |

### Immunohistochemistry

Using immunohistochemistry, TMA1 was stained with antibodies to PERP (1:100, Tris, Abcam, Cambridge, Mass.) and E-cadherin (1:200, Trilogy, Invitrogen, Camarillo, Calif.). TMA2 was stained with PERP only. PERP was graded based on assessment of staining intensity and extent of membrane expression. Cytoplasmic only staining was scored as negative. Intensity was scored as strong (3), weak (2), equivocal (1) or negative (0). For the cores of CIS, oral dysplasia, and normal squamous mucosa, extent of staining was scored as full-thickness (membrane staining extending from the surface into the lower-half of the mucosa), partial-thickness (membrane staining involving only the upper half of the mucosa), or negative (complete loss of PERP). For invasive or metastatic SCC, extent of staining was scored as extensive (>50%), patchy (<50%), or negative. E-cadherin was scored based on intensity of membrane staining as strong (3), weak (2), equivocal (1) or negative (O).

### Statistical Analysis

The Statview statistical package was used for statistical analysis. (Computing Resource Center, San Monica, Calif.) Chi square tests were used to correlate marker's staining thickness and/or staining intensity to different pathologic subtypes (benign, CIS, invasive, metastasis). Kaplan Meier survival curves were generated for freedom from local relapse (FFLR) according to the method of Glanz SAS (Primer of applied regression analysis of variance. New York: McGraw-Hill, Inc.; 1990). Logrank statistics were used to compare survival curves (Cox (1972) J. Royal Stat. Soc. 34:187-229).

### Results

### Extent and Intensity of PERP Membrane Staining

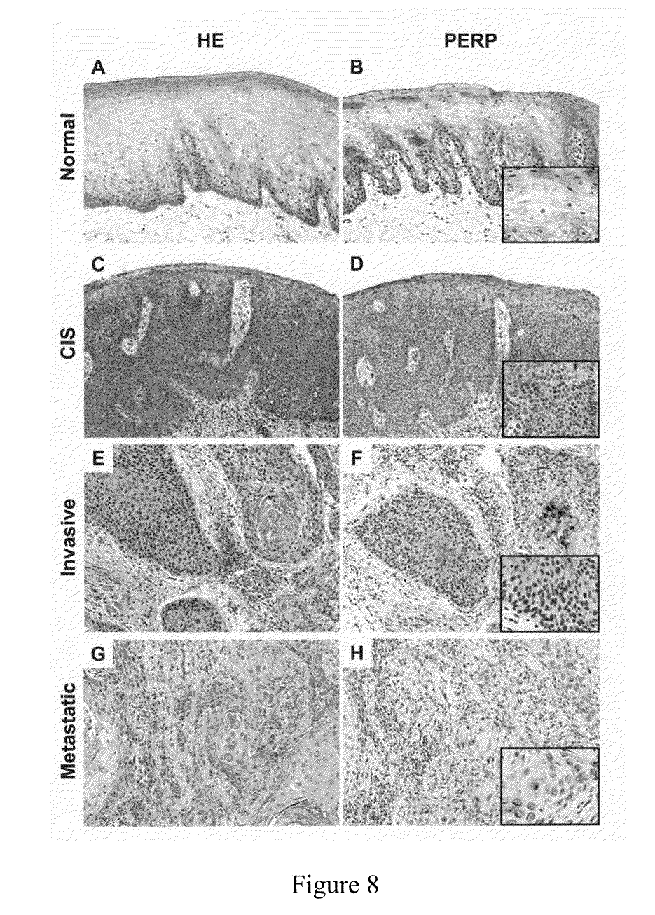

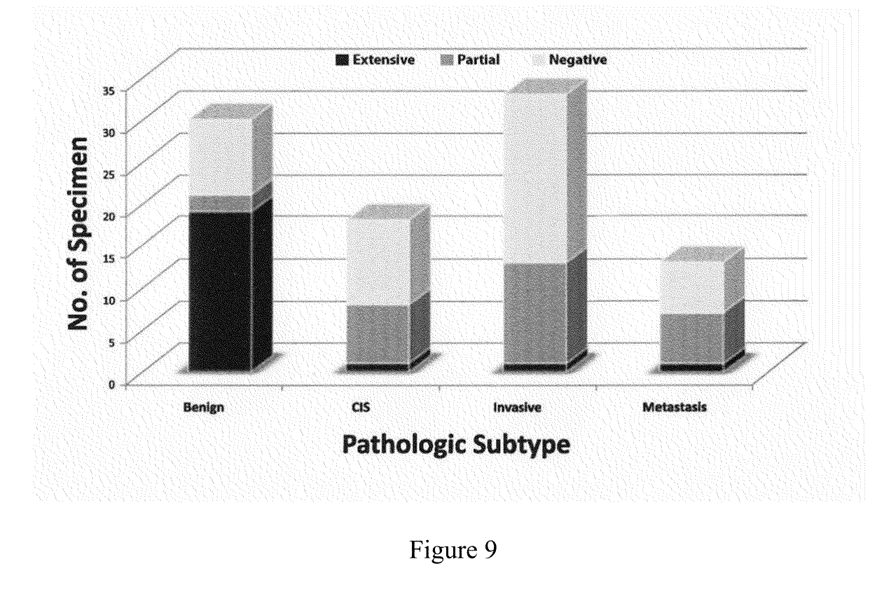

Sixty-three percent of normal squamous mucosa cases exhibit full-thickness membrane staining, extending from the basal layer to the mucosal surface. In contrast, CIS exhibits loss of PERP beginning in the basal layer (see FIG. 8). In 94% CIS cases, there is partial to full thickness loss of PERP. Invasive carcinomas and lymph node metastases also exhibit loss of PERP with patchy or no staining in the majority of cases: 97% invasive carcinomas and 92% nodal metastases. Only rare cases of CIS, invasive carcinoma, and nodal metastasis show full thickness or extensive staining with PERP. The difference in extent of PERP staining between normal and malignant tissue is highly statistically significant (p<0.0001, χ² test; see Table 3).

The intensity of PERP staining also varies significantly between normal and malignant tissue with 97% of adjacent normal mucosa showing strong staining versus 50% of CIS, 23% of invasive SCC and 29% of metastatic tumors (p<0.0001, χ² test; see Table 3).

TABLE 3
Distribution of staining pattern and intensity for different markers

| | | | | | | Invasive | | Nodal | | |
|---|---|---|---|---|---|---|---|---|---|---|
| | Staining Pattern | Benign | | CIS | | Component | | Metastasis | | p-value |
| PERP | Full/extensive | 19/30 | (63%) | 1/18 | (6%) | 1/33 | (3%) | 1/13 | (8%) | <0.0001 |
| extent | Partial/patchy | 2/30 | (7%) | 7/18 | (38%) | 12/33 | (36%) | 6/13 | (46%) | |
| | Negative | 9/30 | (30%) | 10/18 | (56%) | 20/33 | (61%) | 6/13 | (46%) | |
| PERP | Strong | 16/30 | (53%) | 3/18 | (17%) | 7/33 | (24%) | 5/13 | (38%) | <0.0001 |
| Intensity | Weak | 5/30 | (13%) | 5/18 | (28%) | 6/33 | (15%) | 2/13 | (15%) | |
| | Negative | 9/30 | (30%) | 10/18 | (56%) | 20/33 | (61%) | 6/13 | (46%) | |
| E- | Strong | 27/31 | (87%) | 14/17 | (82%) | 23/31 | (74%) | 5/14 | (36%) | 0.008 |
| cadherin | Weak | 4/31 | (13%) | 3/17 | (18%) | 8/31 | (26%) | 8/14 | (57%) | |
| Intensity | Negative | 0/31 | (0%) | 0/17 | (0%) | 0/31 | (0%) | 1/14 | (7%) | |

### E-Cadherin

E-cadherin exhibits extensive membrane staining in normal and malignant squamous epithelium but there is decreased intensity of staining in the metastatic lesions, suggesting that decreased E-cadherin staining is a late event (Table 3). The majority of the normal mucosa (87%), CIS (82%) and invasive SCC specimens (74%) retain strong E-cadherin staining while only 36% of the metastatic lesions displayed strong staining (p=0.008, χ² test).

### Validation of PERP on Oral Dysplasia TMA (TMA2)

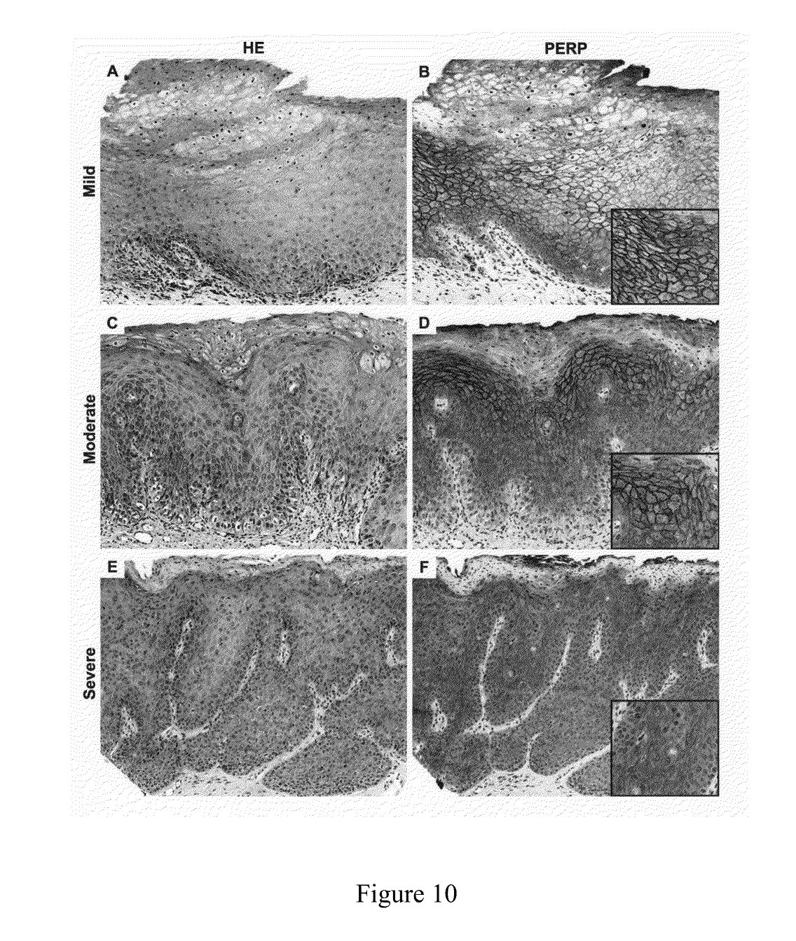

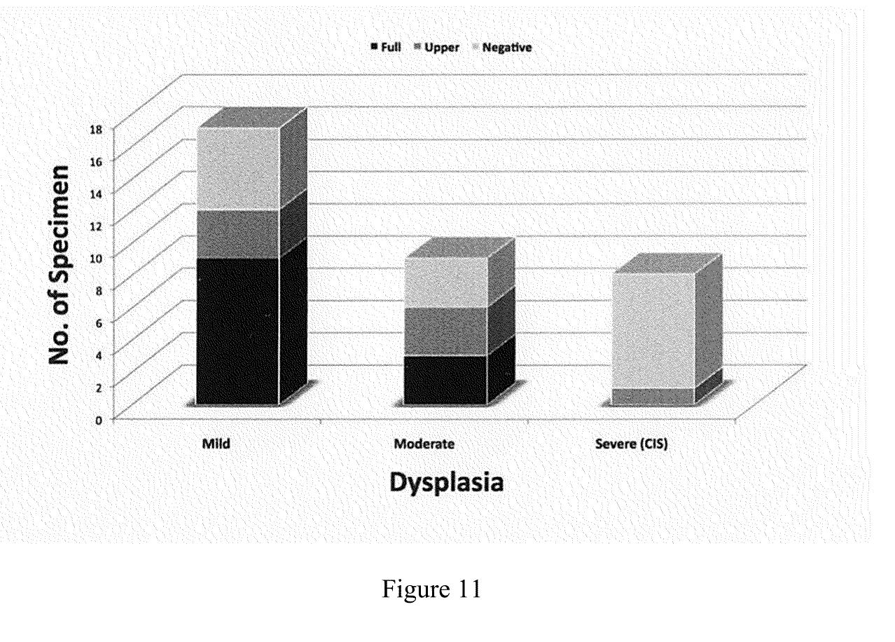

The above observations suggest that PERP loss may be associated with epithelial dysplasia and early invasion. Therefore, we evaluated PERP on an independent TMA that contained mild, moderate and severe oral epithelial dysplasia (TMA2). FIGS. 10 and 11 show extent of PERP staining for different grades of dysplasia. There is a gradual decrease in full thickness PERP staining from mild to moderate and to severe dysplasia. The difference in extent of PERP staining between the 3 groups is statistically significant (p=0.04, χ² test). By contrast, there is no significant difference in intensity of PERP staining when CIS is separated from severe dysplasia and compared with the three groups of dysplasia (p=0.16, data not shown).

### Prognostic Significance of PERP in Invasive SCC (TMA1)

At the latest follow up, 18 patients from the Stanford group (TMA1) have died, 4 are alive with disease and 10 are alive without cancer. Except for two patients who were lost to follow up when they moved outside of the country, all living patients have greater than 2 years of follow up. Twenty patients have failed: local alone in 4, local and nodal in 6, local and distant in 1, nodal alone in 5, nodal and distant in 1, distant alone in 2 and all 3 sites of failure in 1. We recorded the date of local, nodal and distant failure independently since they often occurred sequentially, with local relapse generally occurring first, followed by either nodal or distant relapse.

Since PERP loss appears to be associated with progression from dysplasia to cancer, we hypothesized that decreased PERP expression in the invasive tumor may also correlate with a more clinically aggressive phenotype and thus increased risk of local relapse. For invasive SCC, extent of staining was scored as extensive (>50%), patchy (<50%), or negative. Because there was only one invasive tumor with extensive staining, we grouped it together with those with patchy staining FIG. 12 shows the freedom from local relapse by extent of PERP staining in the invasive tumor component. As shown, 91% of the 14 patients with partial PERP loss retained local control at 5 years compared with only 31% of those with complete PERP loss (n=18, p=0.01). The difference between weak and strong PERP staining was not significant. The 5-year local control rate was 71% for patients with strong PERP staining (n=9), 64% for patients with weak (n=15) and 17% for those with negative staining (n=8). The comparison p-value between these three groups was not statistically significant (p=0.13) but reaches statistical significance (p=0.047) by combining strong and weak into a single positive group and comparing it with the negative group.

PERP staining extent or intensity did not significantly correlate with either nodal or distant relapse though tumors with negative PERP expression generally had a higher rate of nodal and distant recurrence. E-cadherin also did not correlate with local, nodal or distant relapse. Multivariate analysis was not carried out due to the small number of patients.

Discussion

This is the first study to evaluate PERP expression in relation to tumor progression and treatment outcomes in head and neck SCC. We found that in oral cavity cancer, PERP protein loss, specifically a reduction in the extent of mucosal expression occurs early at the transition of mild and moderate dysplasia to severe dysplasia/CIS. This observation was validated on a separate tissue microarray (TMA2) comprising a different set of patients with oral dysplasia. The gradual loss of PERP membrane staining with increasing grade of dysplasia also suggests that PERP loss is an important step in the progression from low grade to higher grades of dysplasia and to cancer. PERP is also potentially useful for identifying patients with low grade dysplasia who are at increased risk of progression to higher grade disease and to invasive carcinoma. However, a prospective study with a larger group of patients would be necessary to test this hypothesis.

PERP loss was also seen with invasive SCC where it correlated with increased local relapse in a subset of patients with known treatment outcomes. Complete PERP loss in the tumor was strongly associated with a high risk of local relapse compared to those that retained partial PERP expression. Loss of PERP was not associated with any aggressive clinical parameter such as larger tumor, less differentiated histologic grade (data not shown), but this may be due to the small patient sample size. Unfortunately, the surgical margin status was not available for this patient group. However, the fact that PERP loss marked the transition of mild and moderate dysplasia to severe dysplasia and was associated with increased risk of relapse suggests that it may serve a role as a molecular tool for evaluating surgical resection margins. Additional studies will be needed to evaluate this potential application of PERP. In contrast, E-cadherin loss appears to occur at a later point in SCC progression, at the time of nodal metastasis. These findings suggest a potential role for PERP as a tumor suppressor gene in the development of keratinizing SCCs, originating in the oral mucosa, as is the case in the skin (Beaudry et al. (2010) PLoS Genet. 6(10):e1001168. PMCID: 2958815). This is the first study that links PERP loss as a marker of early progression and prognosis in oral cavity and HPV(−) OP SCC.

While the preferred embodiments of the invention have been illustrated and described, it will be appreciated that various changes can be made therein without departing from the spirit and scope of the invention.

1. A method for diagnosing cancer or dysplasia in a subject, the method comprising:
a) obtaining a biological sample from a subject suspected of having cancer or dysplasia,
b) measuring the amount of PERP in the biological sample derived from the subject, and
c) analyzing the amount of PERP in conjunction with reference value ranges for PERP, wherein decreased expression of PERP in the biological sample compared to the amount of PERP in a control sample from a normal subject indicates that the subject has cancer or dysplasia.

2. The method of claim 1, further comprising measuring the amount of one or more biomarkers selected from the group consisting of plakoglobin, desmoglein 1/3, and E-cadherin, and analyzing the amounts of the biomarkers in conjunction with respective reference value ranges for the biomarkers.

3. The method of claim 2, comprising measuring the amount of PERP, plakoglobin, and desmoglein 1/3.

4. The method of claim 1, further comprising measuring the amount of one or more biomarkers selected from the group consisting of interleukin 1 family member 6 (IL1f6), S100a9, chitinase 3-like 1 (Chi311), chemokine ligand 20 (Ccl20), and interleukin-22 receptor (IL22ra), and analyzing the amounts of the biomarkers in conjunction with respective reference value ranges for the biomarkers.

5. The method of claim 4, comprising measuring the amount of PERP, IL1f6, S100a9, Chi311, Ccl20, and IL22ra.

6. The method of claim 1, wherein the biological sample is a biopsy comprising cells from a tumor.

7. The method of claim 1, wherein the biological sample is a biopsy comprising cancer in situ.

8. The method of claim 1, wherein the biological sample is a biopsy comprising dysplastic cells.

9. The method of claim 1, wherein the amount of PERP loss is correlated with the stage of dysplasia or cancer by comparison of the amount of PERP in the biological sample to reference value ranges for PERP.

10. The method of claim 9, wherein the amount of PERP loss is correlated with a diagnosis of mild to moderate dysplasia.

11. The method of claim 9, wherein the amount of PERP loss is correlated with a diagnosis of severe dysplasia.

12. The method of claim 9, wherein the amount of PERP loss is correlated with a diagnosis of cancer in situ.

13. The method of claim 9, wherein the amount of PERP loss is correlated with a diagnosis of invasive cancer.

14. The method of claim 1, wherein the cancer is selected from the group consisting of skin squamous cell carcinoma, oral cavity squamous cell carcinoma, head squamous cell carcinoma, neck squamous cell carcinoma, and esophageal squamous cell carcinoma.

15. The method of claim 1, wherein the subject is a human being.

16. The method of claim 1, wherein measuring the amount of PERP in the biological sample comprises performing immunohistochemistry, an enzyme-linked immunosorbent assay (ELISA), a radioimmunoassay (RIA), an immunofluorescent assay (IFA), a sandwich assay, magnetic capture, microsphere capture, a Western Blot, flow cytometry, or mass spectrometry.

17. The method of claim 16, wherein the biological sample comprises desmosomes.

18. The method of claim 16, wherein measuring the amount of PERP comprises contacting an antibody with the PERP, wherein the antibody specifically binds to the PERP, or a fragment thereof containing an antigenic determinant of the PERP.

19. The method of claim 18, wherein the antibody is selected from the group consisting of a monoclonal antibody, a polyclonal antibody, a chimeric antibody, a recombinant fragment of an antibody, an Fab fragment, an Fab′ fragment, an F(ab′)₂ fragment, an Fᵥ fragment, and an scFᵥ fragment.

20. A method for diagnosing dysplasia in a subject, the method comprising:
a) obtaining a biological sample from a subject suspected of having dysplasia,
b) measuring the amount of PERP in the biological sample derived from the subject, and
c) analyzing the amount of PERP in conjunction with reference value ranges for PERP, wherein the reference value ranges are determined by analyzing the amounts of PERP in biological samples derived from subjects with different grades of dysplasia, wherein the amount of PERP in the biological sample is correlated with the grade of dysplasia.

21. A method for determining the prognosis of a subject diagnosed with dysplasia, the method comprising:
a) obtaining a biological sample from the subject,
b) measuring the amount of PERP in the biological sample derived from the subject, wherein complete loss of PERP indicates that the subject is at high risk of developing cancer.

22. A method for determining the prognosis of a subject diagnosed with cancer, the method comprising:
a) obtaining a biological sample from the subject,
b) measuring the amount of PERP in the biological sample derived from the subject, wherein complete loss of PERP indicates that the subject is at high risk of local relapse.

23. The method of claim 22, wherein the biological sample is a biopsy comprising cells from a tumor.

24. The method of claim 22, wherein the biological sample is a biopsy comprising cancer in situ.

25. A method for evaluating the effect of an agent for treating cancer or dysplasia in a subject, the method comprising: analyzing the amount of PERP in samples derived from the subject before and after the subject is treated with said agent, in conjunction with respective reference value ranges for PERP.

26. A method for monitoring the efficacy of a therapy for treating cancer or dysplasia in a subject, the method comprising: analyzing the amount of PERP in samples derived from the subject before and after the subject undergoes said therapy, in conjunction with respective reference value ranges for PERP.

27. A biomarker panel for diagnosing dysplasia or cancer comprising PERP and one or more biomarkers selected from the group consisting of plakoglobin, desmoglein 1/3, E-cadherin, interleukin 1 family member 6 (IL1f6), S100a9, chitinase 3-like 1 (Chi311), chemokine ligand 20 (Ccl20), and interleukin-22 receptor (IL22ra).

28. The biomarker panel of claim 27 comprising PERP, plakoglobin, and desmoglein 1/3.

29. The biomarker panel of claim 27, comprising PERP, interleukin 1 family member 6 (IL1f6), S100a9, chitinase 3-like 1 (Chi311), chemokine ligand 20 (Ccl20), and interleukin-22 receptor (IL22ra).

30. The biomarker panel of claim 27 comprising PERP, plakoglobin, desmoglein 1/3, interleukin 1 family member 6 (IL1f6), S100a9, chitinase 3-like 1 (Chi311), chemokine ligand 20 (Ccl20), and interleukin-22 receptor (IL22ra).

31. A kit comprising agents for measuring the amount of PERP in a biological sample and instructions for using the kit to diagnose dysplasia or cancer.

32. The kit of claim 31, wherein the agents comprise at least one antibody that specifically binds to PERP.

33. The kit of claim 31, further comprising one or more control reference samples.

34. The kit of claim 31, further comprising information, in electronic or paper form, comprising instructions to correlate the detected amount of PERP with cancer or dysplasia.

35. The kit of claim 31, further comprising reagents for performing immunohistochemistry on the biological sample.

36. The kit of claim 31, further comprising agents for measuring the amount of one or more biomarkers selected from the group consisting of plakoglobin, desmoglein 1/3, E-cadherin, interleukin 1 family member 6 (IL1f6), S100a9, chitinase 3-like 1 (Chi311), chemokine ligand 20 (Ccl20), and interleukin-22 receptor (IL22ra).
